(19) United States
(12) Patent Application Publication
(10) Pub. No.: US 2019/0135857 A1
(43) Pub. Date: May 9, 2019

(54) TRITERPENOID INHIBITORS OF HUMAN IMMUNODEFICIENCY VIRUS REPLICATION

(71) Applicant: VIIV HEALTHCARE UK (NO. 5) LIMITED, Brentford, Middlesex (GB)

(72) Inventors: Brian Lee VENABLES, Wallingford, CT (US); Jacob SWIDORSKI, Wallingford, CT (US); Ny SIN, Wallingford, CT (US); Alicia REGUEIRO-REN, Wallingford, CT (US)

(21) Appl. No.: 16/306,083

(22) PCT Filed: Jun. 28, 2017
(86) PCT No.: PCT/IB2017/053880

§ 371 Date: Nov. 30, 2018

Related U.S. Application Data
(60) Provisional application No. 62/356,840, filed on Jun. 30, 2016.

(51) Int. Cl. C07J 63/00; A61P 31/18

(52) U.S. Cl. CPC C07J 63/008; A61P 31/18

(57) ABSTRACT

Compounds having drug and bio-affecting properties, their pharmaceutical compositions and methods of use are set forth. In particular, triterpenoid compounds that possess unique antiviral activity are provided as HIV maturation inhibitors, as represented by compounds of Formula I:

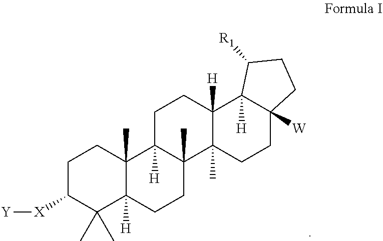

These compounds are useful for the treatment of HIV and AIDS.

### FIELD OF THE INVENTION

The invention relates to compounds, compositions, and methods for the treatment of human immunodeficiency virus (HIV) infection. More particularly, the invention provides novel triterpenoid compounds as inhibitors of HIV, pharmaceutical compositions containing such compounds, and methods for using these compounds in the treatment of HIV infection. The invention also relates to methods for making the compounds hereinafter described.

### BACKGROUND OF THE INVENTION

Acquired immunodeficiency syndrome (AIDS) is the result of infection by HIV. HIV infection remains a major medical problem, with an estimated 45-50 million people infected worldwide at the end of 2011, 3.3 million of them under the age of 15. In 2011, there were 2.5 million new infections, and 1.7 million deaths from complications due to HIV/AIDS.

Current therapy for HIV-infected individuals consists of a combination of approved anti-retroviral agents. Over two dozen drugs are currently approved for HIV infection, either as single agents or as fixed dose combinations or single tablet regimens, the latter two containing 2-4 approved agents. These agents belong to a number of different classes, targeting either a viral enzyme or the function of a viral protein during the virus replication cycle. Thus, agents are classified as either nucleotide reverse transcriptase inhibitors (NRTIs), non-nucleotide reverse transcriptase inhibitors (NNRTIs), protease inhibitors (PIs), integrase inhibitors (INIs), or entry inhibitors (one, maraviroc, targets the host CCR5 protein, while the other, enfuvirtide, is a peptide that targets the gp41 region of the viral gp160 protein). In addition, a pharmacokinetic enhancer with no antiviral activity, i.e., cobicistat, available from Gilead Sciences, Inc. under the tradename TYBOST™ (cobicistat) tablets, has recently been approved for use in combinations with certain antiretroviral agents (ARVs) that may benefit from boosting.

Despite the armamentarium of agents and drug combinations, there remains a medical need for new anti-retroviral agents, due in part to the need for chronic dosing to combat infection. Significant problems related to long-term toxicities are documented, creating a need to address and prevent these co-morbidities (e.g. CNS, CV/metabolic, renal disease). Also, increasing failure rates on current therapies continue to be a problem, due either to the presence or emergence of resistant strains or to non-compliance attributed to drug holidays or adverse side effects. For example, despite therapy, it has been estimated that 63% of subjects receiving combination therapy remained viremic, as they had viral loads >500 copies/mL (Oette, M, Kaiser, R, Daumer, M, et al. Primary HIV Drug Resistance and Efficacy of First-Line Antiretroviral Therapy Guided by Resistance Testing. J Acq Imm Def Synd 2006; 41(5):573-581). Among these patients, 76% had viruses that were resistant to one or more classes of antiretroviral agents. As a result, new drugs are needed that are easier to take, have high genetic barriers to the development of resistance and have improved safety over current agents. In this panoply of choices, novel MOAs that can be used as part of the preferred highly active antiretroviral therapy (HAART) regimen can still have a major role to play since they should be effective against viruses resistant to current agents.

Certain therapeutic compounds are disclosed in WO 2013/006738, WO 2014/110298, and WO 2014/134566.

What is now needed in the art are additional compounds which are novel and useful in the treatment of HIV. Additionally, these compounds may desirably provide advantages for pharmaceutical uses, for example, with regard to one or more of their mechanisms of action, binding, inhibition efficacy, target selectivity, solubility, safety profiles, or bioavailability. Also needed are new formulations and methods of treatment which utilize these compounds.

### SUMMARY OF THE INVENTION

The invention encompasses compounds of Formula I, including pharmaceutically acceptable salts thereof, as well as pharmaceutical compositions, and their use in inhibiting HIV and treating those infected with HIV or AIDS.

In one aspect of the invention, there is provided a compound of Formula I, including pharmaceutically acceptable salts thereof:

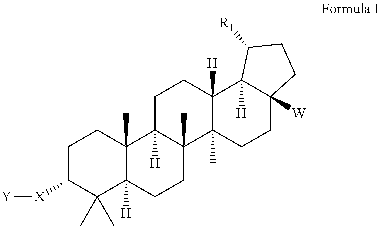

wherein R₁ is isopropenyl or isopropyl;
X is a phenyl or heteroaryl ring substituted with A, wherein A is at least one member selected from —H, -halo, -hydroxyl, —C₁₋₆ alkyl, —C₁₋₆ alkoxy, and —COOR₂;
R₂ is —H, —C₁₋₆ alkyl, -alkylsubstituted C₁₋₆ alkyl or -arylsubstituted C₁₋₆ alkyl;
Y is selected from —COOR₂, —C(O)NR₂SO₂R₃, —C(O)NHSO₂NR₂R₂, —NR₂SO₂R₂, —SO₂NR₂R₂, —C₃₋₆ cycloalkyl-COOR₂, —C₂₋₆ alkenyl-COOR₂, —C₂₋₆ alkynyl-COOR₂, —C₁₋₆ alkyl-COOR₂, —NHC(O)(CH₂)ₙ—COOR₂, —SO₂NR₂C(O)R₂, -tetrazole, and —CONHOH,
wherein n=1-6;
R₃ is —C₁₋₆ alkyl or -alkylsubstituted C₁₋₆ alkyl;
W is selected from —CH₂OR₂, —COOR₂, —NR₄R₅, —CONR₂₆R₂₇, —CH₂NR₂₆R₂₇, —NR₄COR₆, —NR₄C(O)NR₄R₅, and —NR₄COOR₆;
R₄ is selected from —H, —C₁₋₆ alkyl, —C₁₋₆ alkyl-C(OR₃)₂—C₃₋₆ cycloalkyl, —C₁₋₆ substituted alkyl, —C₁₋₆ alkyl-C₃₋₆ cycloalkyl, —C₁₋₆ alkyl-Q₁, —C₁₋₆ alkyl-C₃₋₆ cycloalkyl-Q₁, aryl, heteroaryl, substituted heteroaryl, —COR₆, —COCOR₆, —SO₂R₇, —SO₂NR₂R₂,
wherein Q₁ is selected from C₃₋₁₀ carbocycle, substituted C₃₋₁₀ carbocycle, C₃₋₁₀ heterocycle, substituted C₃₋₁₀ hetereocycle, aryl, heteroaryl, substituted heteroaryl, halogen, —CF₃, —OR₂, —COOR₂, —NR₈R₉, —CONR₁₀R₁₁ and —SO₂R₇;
R₅ is selected from —H, —C₁₋₆ alkyl, —C₃₋₆ cycloalkyl, —C₁₋₆ alkylsubstituted alkyl, —C₁₋₆ alkyl-NR₈R₉, —COR₆, —COCOR₆, —SO₂R₇ and —SO₂NR₂R₂;
with the proviso that only one of R₄ or R₅ can be selected from —COR₆, —COCOR₆, —SO₂R₇ and —SO₂NR₂R₂;
R₆ is selected from —H, —C₁₋₆ alkyl, —C₁₋₆ alkyl-substitutedalkyl, —C₃₋₆ cycloalkyl, —C₃₋₆ substitutedcycloalkyl-Q₂, —C₁₋₆ alkyl-Q₂, —C₁₋₆ alkyl-substitutedalkyl-Q₂, —C₃₋₆ cycloalkyl-Q₂, aryl-Q₂, —NR₁₃R₁₄, and —OR₁₅;
wherein Q₂ is selected from C₃₋₁₀ carbocycle, substituted C₃₋₁₀ carbocycle, C₃₋₁₀ heterocycle, substituted C₃₋₁₀ hetereocycle, aryl, heteroaryl, substituted heteroaryl, —OR₂, —COOR₂, —NR₈R₉, SO₂R₇, —CONHSO₂R₃, and —CONHSO₂NR₂R₂;
R₇ is selected from —C₁₋₆ alkyl, —C₁₋₆ substituted alkyl, —C₃₋₆ cycloalkyl, aryl, and heteroaryl;
R₈ and R₉ are independently selected from —H, —C₁₋₆ alkyl, —C₁₋₆ substituted alkyl, aryl, heteroaryl, substituted aryl, substituted heteroaryl, —C₁₋₆ alkyl-Q₂, and —COOR₃,
or R₈ and R₉ are taken together with the adjacent N to form a cycle selected from:

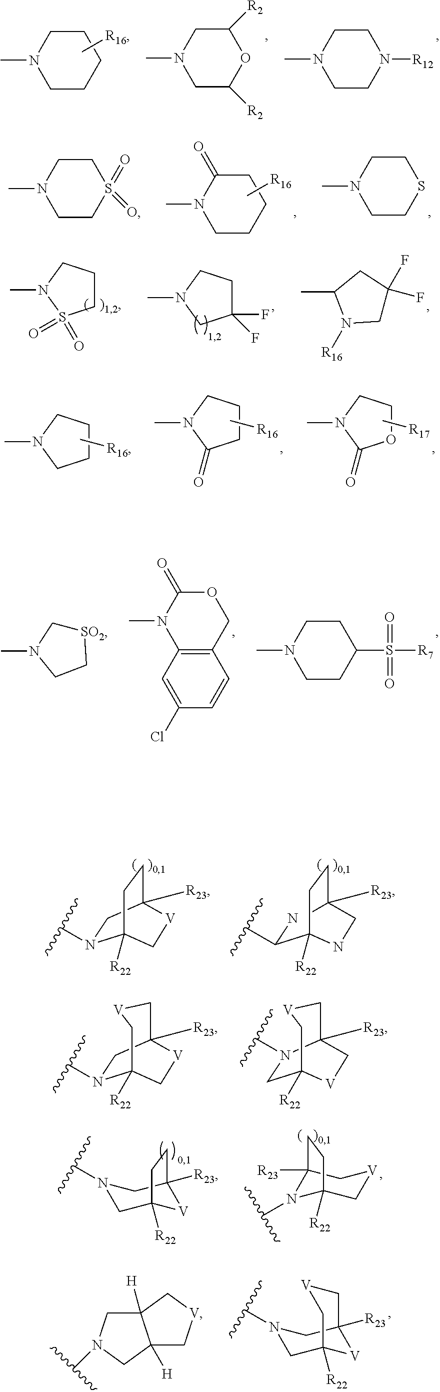

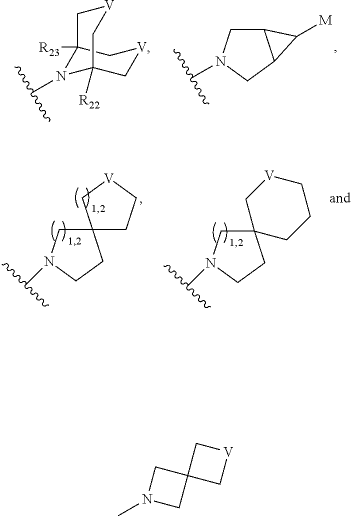

V is selected from —CR₂₄R₂₅, —SO₂, —O and —NR₁₂;
M is selected from —CHR₂₄R₂₅, —NR₂₆R₂₇, —SO₂R₇, —SO₂NR₃R₃ and —OH;
with the proviso that only one of R₈ or R₉ can be —COOR₃;
R₁₀ and R₁₁ are independently selected from —H, —C₁₋₆ alkyl, —C₁₋₆ substituted alkyl and —C₃₋₆ cycloalkyl,
or R₁₀ and R₁₁ are taken together with the adjacent N to form a cycle such as

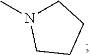

R₁₂ is selected from —C₁₋₆ alkyl, —C₁₋₆ alkyl-OH; —C₁₋₆ alkyl, —C₁₋₆ substituted alkyl, —C₃₋₆ cycloalkyl, —COR₇, —COONR₂₂R₂₃, —SOR₇, and —SONR₂₄R₂₅;
R₁₃ and R₁₄ are independently selected from —H, —C₁₋₆ alkyl, —C₃₋₆ cycloalkyl, —C₁₋₆ substituted alkyl, —C₁₋₆ alkyl-Q₃, —C₁₋₆ alkyl-C₃₋₆ cycloalkyl-Q₃ and C₁₋₆ substituted alkyl-Q₃,
or R₁₃ and R₁₄ are taken together with the adjacent N to form a cycle selected from:

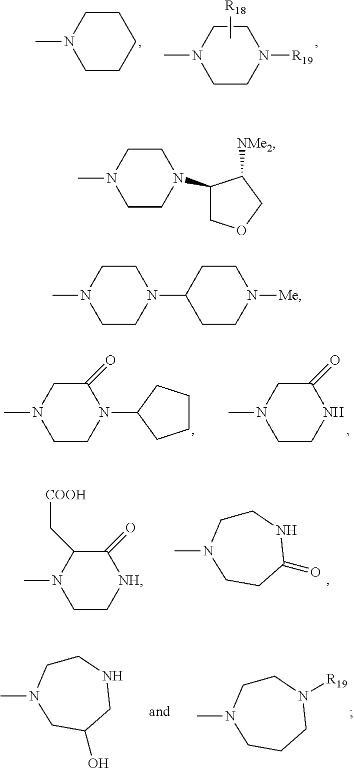

Q₃ is selected from heteroaryl, substituted heteroaryl, —NR₂₀R₂₁, ⁻CONR₂R₂, —COOR₂, —OR₂, and —SO₂R₃;
R₁₅ is selected from —C₁₋₆ alkyl, —C₃₋₆ cycloalkyl, —C₁₋₆ substituted alkyl, —C₁₋₆ alkyl-Q₃, —C₁₋₆ alkyl-C₃₋₆ cycloalkyl-Q₃ and —C₁₋₆ substituted alkyl-Q₃;
R₁₆ is selected from —H, —C₁₋₆ alkyl, —NR₂R₂, and —COOR₃;
R₁₇ is selected from —H, —C₁₋₆ alkyl, —COOR₃, and aryl;
R₁₈ is selected from —COOR₂ and —C₁₋₆ alkyl-COOR₂;
R₁₉ is selected from —H, —C₁₋₆ alkyl, —C₁₋₆ alkyl-Q₄, —COR₃, —COOR₃,
wherein Q₄ is selected from —NR₂R₂ and —OR₂;
R₂₀ and R₂₁ are independently selected from —H, —C₁₋₆ alkyl, —C₁₋₆ substituted alkyl, —C₁₋₆ substituted alkyl-OR₂, and —COR₃,
or R₂₀ and R₂₁ are taken together with the adjacent N to form a cycle selected from

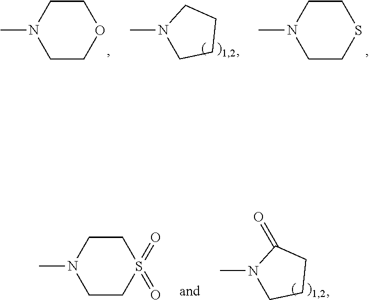

with the proviso that only one of R₂₀ or R₂₁ can be —COR₃;
R₂₂ and R₂₃ are independently selected from H, —C₁₋₆ alkyl, —C₁₋₆ substituted alkyl, and —C₁₋₆ cycloalkyl,
or R₂₂ and R₂₃ are taken together with the adjacent N to form a cycle selected from

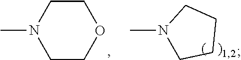

R₂₄ and R₂₅ are independently from the group of H, —C₁₋₆ alkyl, —C₁₋₆ substituted alkyl, —C₁₋₆ alkyl-Q₅, —C₁₋₆ cycloalkyl, aryl, substituted aryl, heteroaryl, and substituted heteroaryl, and Q₅ is selected from halogen and SO₂R₃,
R₂₆ and R₂₇ are independently selected from —H, —C₁₋₆ alkyl, —C₁₋₆ substituted alkyl, aryl, heteroaryl, substituted aryl, substituted heteroaryl, —C₁₋₆ alkyl-Q₂,
or R₂₆ and R₂₇ are taken together with the adjacent N to form a cycle selected from:

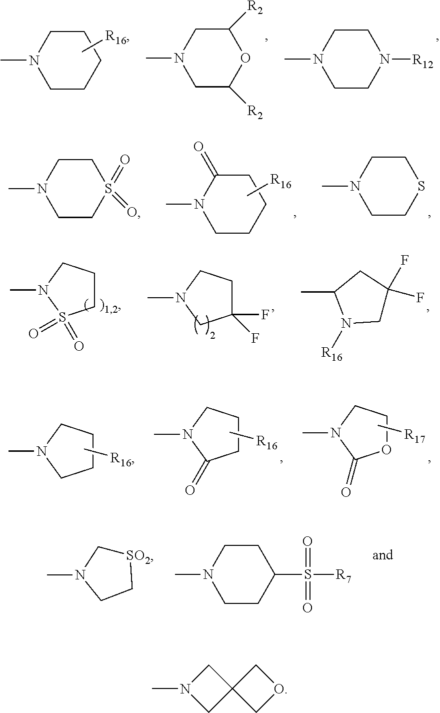

With respect to the compounds of Formula I, the “—X—Y” substituent at the C-3 position, has the indicated stereochemistry as shown below with the “dotted wedge”. In contrast, many substituted triterpenoid compounds have the opposite stereochemistry at the C-3 position, which would instead be indicated by a “solid wedge”.

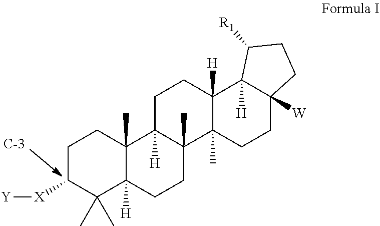

In an aspect of the invention, there is provided a composition useful for treating HIV infection comprising a therapeutic amount of a compound of Formula I and a pharmaceutically acceptable carrier. In an aspect of the invention, the composition further comprises a therapeutically effective amount at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors, and a pharmaceutically acceptable carrier. In an aspect of the invention, the other agent is dolutegravir.

In an aspect of the invention, there is provided a method for treating HIV infection comprising administering a therapeutically effective amount of a compound of Formula I, or a pharmaceutically acceptable salt thereof, to a patient in need thereof. In an aspect of the invention, the method further comprises administering a therapeutically effective amount of at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors. In an aspect of the invention, the other agent is dolutegravir. In an aspect of the invention, the other agent is administered to the patient prior to, simultaneously with, or subsequently to the compound of Formula I.

Also provided as part of the invention are one or more methods for making the compounds of the invention.

The present invention is directed to these, as well as other important ends, hereinafter described.

### DETAILED DESCRIPTION OF THE INVENTION

The singular forms “a”, “an”, and “the” include plural reference unless the context dictates otherwise.

Unless otherwise expressly set forth elsewhere in the application, the following terms shall have the following meanings:

“Alkenyl” means a straight or branched alkyl group comprised of 2 to 10 carbons with at least one double bond and optionally substituted with 0-3 halo or alkoxy group.

“Alkenyloxy” means an alkenyl group attached to the parent structure by an oxygen atom.

“Alkoxy” means an alkyl group attached to the parent structure by an oxygen atom.

“Alkoxycarbonyl” means an alkoxy group attached to the parent structure by a carbonyl moiety.

“Alkyl” means a straight or branched saturated hydrocarbon comprised of 1 to 10 carbons, and preferably 1 to 6 carbons.

“Alkylthioxy” means an alkyl group attached to the parent structure through a sulfur atom.

“Alkynyl” means a straight or branched alkyl group comprised of 2 to 10 carbons, preferably 2 to 6 carbons, containing at least one triple bond and optionally substituted with 0-3 halo or alkoxy group.

“Aryl” mean a carbocyclic group comprised of 1-3 rings that are fused and/or bonded and at least one or a combination of which is aromatic. The non-aromatic carbocyclic portion, where present, will be comprised of C₃ to C₇ alkyl group. Examples of aromatic group include, but are not limited to, phenyl, biphenyl, cyclopropylphenyl, indane, naphthalene, and tetrahydronaphthalene. The aryl group can be attached to the parent structure through any substitutable carbon atom in the group.

“Arylalkyl” is a C₁-C₅ alkyl group attached to 1 to 2 aryl groups and linked to the parent structure through the alkyl moiety. Examples include, but are not limited to, —(CH₂)ₙPh with n=1-5, —CH(CH₃)Ph, —CH(Ph)₂.

“Aryloxy” is an aryl group attached to the parent structure by oxygen.

“Azaindole” means one of the “CH” moieties in the 6-member ring of an indole is substituted with a nitrogen atom.

“Azaindoline” means one of the aromatic “CH” moieties of an indoline is substituted with a nitrogen atom.

“Azatetrahydroquinoline” means any aromatic CH moiety of tetrahydroquinoline is substituted with a nitrogen atom.

“Benzyloxy” means a benzyl group is attached to the parent structure through an oxygen atom. The phenyl group of the benzyl moiety could be optionally substituted by 1-3 moieties independently selected from alkyl, alkoxy, halo, haloalkyl, haloalkoxy and cyano.

“Cₓ-C_{y}” notation indicates a structural element comprised of carbons numbering between ‘x’ and ‘y’. For example, “C₅-C₁₀ bicycloalkyl” means a bicyclic ring system comprised of 5 to 10 carbons, where the rings are attached in a fused, spiro or bridged manner; an example of C₅-C₁₀ bicycloalkyl include, but is not limited to, bicyclo[2.2.2]octane. Similarly, “C₃-C₄ cycloalkyl” is a subset of monocyclic ring system comprised of 3 to 4 carbons.

“Cycloalkyl” means a monocyclic ring system comprised of 3 to 7 carbons.

“Cyano” refers to —CN.

“Diazaindole” means any two “CH” moieties in the 6-member ring of an indole are substituted with nitrogen atoms.

“Diazaindoline” means any two aromatic “CH” moieties of an indoline are substituted with a nitrogen atom.

“Diazatetrahydroquinoline” means any two aromatic CH moieties of tetrahydroquinoline are substituted with nitrogen atoms.

“Halo” or “halogen” refers to —F, —Cl, —Br, or —I.

“Haloalkyl” means an alkyl group substituted by any combination of one to six halogen atoms.

“Haloalkoxy” or “Haloalkyloxy” means a haloalkyl group attached to the parent structure through an oxygen atom.

“Hydroxy” refers to —OH.

“Heteroaryl” is a subset of heterocyclic group as defined below and is comprised of 1-3 rings where at least one or a combination of which is aromatic and that the aromatic group contains at least one atom chosen from a group of oxygen, nitrogen or sulfur.

“Heterocyclyl or heterocyclic” means a cyclic group of 1-3 rings comprised of carbon and at least one other atom selected independently from oxygen, nitrogen and sulfur. The rings could be bridged, fused and/or bonded, through a direct or spiro attachment, with the option to have one or a combination thereof be aromatic. Examples include, but are not limited to, azaindole, azaindoline, azetidine, benzimidazole, bezodioxolyl, benzoisothiazole, benzothiazole, benzothiadiazole, benzothiophene, benzoxazole, carbazole, chroman, dihalobezodioxolyl, dihydrobenzofuran, dihydrobenzo[1,4]oxazine, 1,3-dihydrobenzo[c]thiophene 2,2-dioxide, 2,3-dihydrobenzo[d]isothiazole 1,1-dioxide, 3,4-dihydro-2H-pyrido[3,2-b][1,4]oxazine, 2,3-dihydro-1H-pyrrolo[3,4-c]pyridine and its regioisomeric variants, 6,7-dihydro-5H-pyrrolo[2,3-b]pyrazine and its regioisomeric variants, furanylphenyl, imidazole, imidazo[1,2-a]pyridine, indazole, indole, indoline, isoquinoline, isoquinolinone, isothiazolidine 1,1-dioxide, morpholine, 2-oxa-5-azabicyclo[2.2.1]heptane, oxadiazole-phenyl, oxazole, phenylaztidine, phenylindazole, phenylpiperidine, phenylpiperizine, phenyloxazole, phenylpyrrolidine, piperidine, pyridine, pyridinylphenyl, pyridinylpyrrolidine, pyrimidine, pyrimidinylphenyl, pyrrazole-phenyl, pyrrolidine, pyrrolidin-2-one, 1H-pyrazolo[4,3-c]pyridine and its regioisomeric variants, pyrrole, 5H-pyrrolo[2,3-b]pyrazine, 7H-pyrrolo[2,3-d]pyrimidine and its regioisomeric variants, quinazoline, quinoline, quinoxaline, tetrahydroisoquinoline, 1,2,3,4-tetrahydro-1,8-naphthyridine, tetrahydroquinoline, 4,5,6,7-tetrahydrothieno[3,2-c]pyridine, 1,2,5-thiadiazolidine 1,1-dioxide, thiophene, thiophenylphenyl, triazole, or triazolone. Unless otherwise specifically set forth, the heterocyclic group can be attached to the parent structure through any suitable atom in the group that results in a stable compound.

It is understood that a subset of the noted heterocyclic examples encompass regioisomers. For instance, “azaindole” refers to any of the following regioisomers: 1H-pyrrolo[2,3-b]pyridine, 1H-pyrrolo[2,3-c]pyridine, 1H-pyrrolo[3,2-c]pyridine, and 1H-pyrrolo[3,2-b]pyridine. In addition the “regioisomer variants” notation as in, for example, “5H-pyrrolo[2,3-b]pyrazine and its regioisomeric variants” would also encompass 7H-pyrrolo[2,3-d]pyrimidine, 7H-pyrrolo[2,3-c]pyridazine, 1H-pyrrolo[2,3-d]pyridazine, 5H-pyrrolo[3,2-c]pyridazine, and 5H-pyrrolo[3,2-d]pyrimidine. Similarly, 6,7-dihydro-5H-pyrrolo[2,3-b]pyrazine and its regioisomeric variants would encompass 6,7-dihydro-5H-pyrrolo[2,3-d]pyrimidine and 6,7-dihydro-5H-pyrrolo[2,3-c]pyridazine. It is also understood that the lack of “regioisomeric variants” notation does not in any way restrict the claim scope to the noted example only.

“Heterocyclylalkyl” is a heterocyclyl moiety attached to the parent structure through C₁-C₅ alkyl group. Examples include, but are not limited to, —(CH₂)ₙ—R^{Z} or —CH(CH₃)—(R^{Z}) where n=1-5 and that R^{Z} is chosen from benzimidazole, imidazole, indazole, isooxazole, phenyl-pyrazole, pyridine, quinoline, thiazole, triazole, triazolone, oxadiazole.

“Triterpene” or “triterpenoid” means a class of compounds based on three terpene units, which are in turn each based on two isoprene units. Triterpenes exist in a large variety of structures and can be broadly divided according to the number of rings present. The triterpenoids of the present invention are in general pentacyclic structures, i.e. having five rings.

“Tetrahydroquinoline” means 1,2,3,4-tetrahydroquinoline.

Substituents which are illustrated by chemical drawing to bond at variable positions on a multiple ring system (for example a bicyclic ring system) are intended to bond to the ring where they are drawn to append. Parenthetic and multiparenthetic terms are intended to clarify bonding relationships to those skilled in the art. For example, a term such as ((R)alkyl) means an alkyl substituent further substituted with the substituent R.

Those terms not specifically set forth herein shall have the meaning which is commonly understood and accepted in the art.

The invention includes all pharmaceutically acceptable salt forms of the compounds. Pharmaceutically acceptable salts are those in which the counter ions do not contribute significantly to the physiological activity or toxicity of the compounds and as such function as pharmacological equivalents. These salts can be made according to common organic techniques employing commercially available reagents. Some anionic salt forms include acetate, acistrate, besylate, bromide, chloride, citrate, fumarate, glucouronate, hydrobromide, hydrochloride, hydroiodide, iodide, lactate, maleate, mesylate, nitrate, pamoate, phosphate, succinate, sulfate, tartrate, tosylate, and xinofoate. Some cationic salt forms include ammonium, aluminum, benzathine, bismuth, calcium, choline, diethylamine, diethanolamine, lithium, magnesium, meglumine, 4-phenylcyclohexylamine, piperazine, potassium, sodium, tromethamine, and zinc.

Some of the compounds of the invention exist in stereoisomeric forms. The invention includes all stereoisomeric forms of the compounds including enantiomers and diastereromers. Methods of making and separating stereoisomers are known in the art. The invention includes all tautomeric forms of the compounds. The invention includes atropisomers and rotational isomers.

The invention is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. Such compounds may have a variety of potential uses, for example as standards and reagents in determining biological activity. In the case of stable isotopes, such compounds may have the potential to favorably modify biological, pharmacological, or pharmacokinetic properties.

For the sake of efficiency some ring structures are shown with a variable number of members in the ring. For example, the following ring substituent

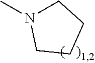

having the parenthetical “( )_{1,2}” is intended to encompass both a single carbon group, —(CH₂)—, and a two carbon group, —(CH₂CH₂)—. The intended ring structures could individually be depicted as:

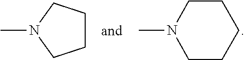

The compounds of the invention also include “prodrugs”. The term “prodrug” as used herein encompasses both the term “prodrug esters” and the term “prodrug ethers”.

The common numbering system for the general core of the pentacyclic triterpenoid compounds of the present invention is shown below for an illustrative triterpene:betulin. This numbering system is in accordance with IUPAC rules.

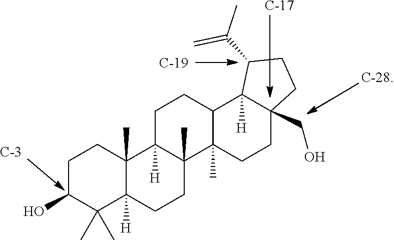

In an aspect of the invention, there is provided a compound of Formula I, including pharmaceutically acceptable salts thereof:

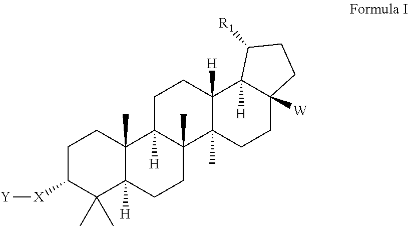

wherein R₁ is isopropenyl or isopropyl;
X is a phenyl or heteroaryl ring substituted with A, wherein A is at least one member selected from —H, -halo, -hydroxyl, —C₁₋₆ alkyl, —C₁₋₆ alkoxy, and —COOR₂;
R₂ is —H, —C₁₋₆ alkyl, -alkylsubstituted C₁₋₆ alkyl or -arylsubstituted C₁₋₆ alkyl;
Y is selected from —COOR₂, —C(O)NR₂SO₂R₃, —C(O)NHSO₂NR₂R₂, —NR₂SO₂R₂, —SO₂NR₂R₂, —C₃₋₆ cycloalkyl-COOR₂, —C₂₋₆ alkenyl-COOR₂, —C₂₋₆ alkynyl-COOR₂, —C₁₋₆ alkyl-COOR₂, —NHC(O)(CH₂)ₙ—COOR₂, —SO₂NR₂C(O)R₂, -tetrazole, and —CONHOH,
wherein n=1-6;
R₃ is —C₁₋₆ alkyl or -alkylsubstituted C₁₋₆ alkyl;
W is selected from —CH₂OR₂, —COOR₂, —NR₄R₅, —CONR₂₆R₂₇, —CH₂NR₂₆R₂₇, —NR₄COR₆, —NR₄C(O)NR₄R₅, and —NR₄COOR₆;
R₄ is selected from —H, —C₁₋₆ alkyl, —C₁₋₆ alkyl-C(OR₃)₂—C₃₋₆ cycloalkyl, —C₁₋₆ substituted alkyl, —C₁₋₆ alkyl-C₃₋₆ cycloalkyl, —C₁₋₆ alkyl-Q₁, —C₁₋₆ alkyl-C₃₋₆ cycloalkyl-Q₁, aryl, heteroaryl, substituted heteroaryl, —COR₆, —COCOR₆, —SO₂R₇, —SO₂NR₂R₂,
wherein Q₁ is selected from C₃₋₁₀ carbocycle, substituted C₃₋₁₀ carbocycle, C₃₋₁₀ heterocycle, substituted C₃₋₁₀ hetereocycle, aryl, heteroaryl, substituted heteroaryl, halogen, —CF₃, —OR₂, —COOR₂, —NR₈R₉, —CONR₁₀R₁₁ and —SO₂R₇;
R₅ is selected from —H, —C₁₋₆ alkyl, —C₃₋₆ cycloalkyl, —C₁₋₆ alkylsubstituted alkyl, —C₁₋₆ alkyl-NR₈R₉, —COR₆, —COCOR₆, —SO₂R₇ and —SO₂NR₂R₂;
with the proviso that only one of R₄ or R₅ can be selected from —COR₆, —COCOR₆, —SO₂R₇ and —SO₂NR₂R₂;
R₆ is selected from —H, —C₁₋₆ alkyl, —C₁₋₆ alkyl-substitutedalkyl, —C₃₋₆ cycloalkyl, —C₃₋₆ substitutedcycloalkyl-Q₂, —C₁₋₆ alkyl-Q₂, —C₁₋₆ alkyl-substitutedalkyl-Q₂, —C₃₋₆ cycloalkyl-Q₂, aryl-Q₂, —NR₁₃R₁₄, and —OR₁₅;
wherein Q₂ is selected from C₃₋₁₀ carbocycle, substituted C₃₋₁₀ carbocycle, C₃₋₁₀ heterocycle, substituted C₃₋₁₀ hetereocycle, aryl, heteroaryl, substituted heteroaryl, —OR₂, —COOR₂, —NR₈R₉, SO₂R₇, —CONHSO₂R₃, and —CONHSO₂NR₂R₂;
R₇ is selected from —C₁₋₆ alkyl, —C₁₋₆ substituted alkyl, —C₃₋₆ cycloalkyl, aryl, and heteroaryl;
R₈ and R₉ are independently selected from —H, —C₁₋₆ alkyl, —C₁₋₆ substituted alkyl, aryl, heteroaryl, substituted aryl, substituted heteroaryl, —C₁₋₆ alkyl-Q₂, and —COOR₃,
or R₈ and R₉ are taken together with the adjacent N to form a cycle selected from:

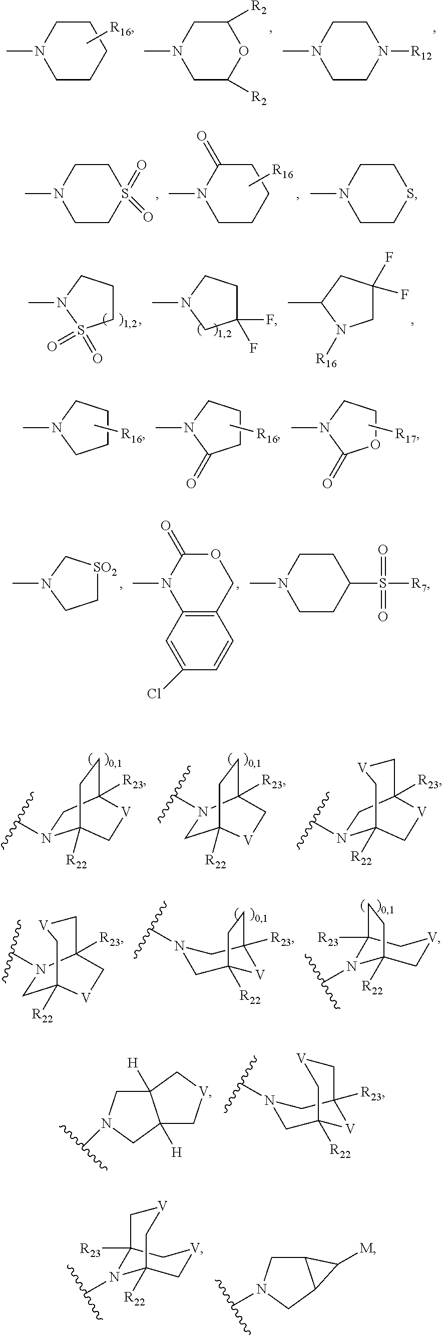

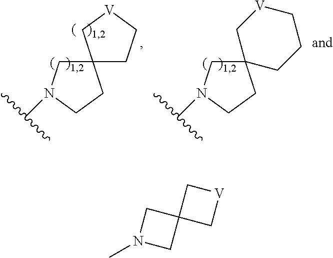

V is selected from —CR₂₄R₂₅, —SO₂, —O and —NR₁₂;
M is selected from —CHR₂₄R₂₅, —NR₂₆R₂₇, —SO₂R₇, —SO₂NR₃R₃ and —OH;
with the proviso that only one of R₈ or R₉ can be —COOR₃;
R₁₀ and R₁₁ are independently selected from —H, —C₁₋₆ alkyl, —C₁₋₆ substituted alkyl and —C₃₋₆ cycloalkyl,
or R₁₀ and R₁₁ are taken together with the adjacent N to form a cycle such as

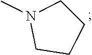

R₁₂ is selected from —C₁₋₆ alkyl, —C₁₋₆ alkyl-OH; —C₁₋₆ alkyl, —C₁₋₆ substituted alkyl, —C₃₋₆ cycloalkyl, —COR₇, —COONR₂₂R₂₃, —SOR₇, and —SONR₂₄R₂₅;
R₁₃ and R₁₄ are independently selected from —H, —C₁₋₆ alkyl, —C₃₋₆ cycloalkyl, —C₁₋₆ substituted alkyl, —C₁₋₆ alkyl-Q₃, —C₁₋₆ alkyl-C₃₋₆ cycloalkyl-Q₃ and C₁₋₆ substituted alkyl-Q₃,
or R₁₃ and R₁₄ are taken together with the adjacent N to form a cycle selected from:

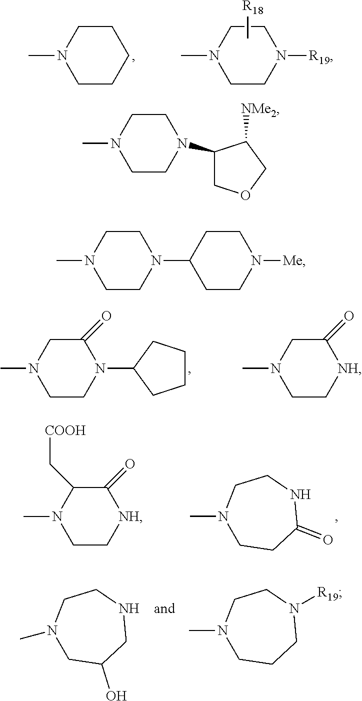

Q₃ is selected from heteroaryl, substituted heteroaryl, —NR₂₀R₂₁, ⁻CONR₂R₂, —COOR₂, —OR₂, and —SO₂R₃;
R₁₅ is selected from —C₁₋₆ alkyl, —C₃₋₆ cycloalkyl, —C₁₋₆ substituted alkyl, —C₁₋₆ alkyl-Q₃, —C₁₋₆ alkyl-C₃₋₆ cycloalkyl-Q₃ and —C₁₋₆ substituted alkyl-Q₃;
R₁₆ is selected from —H, —C₁₋₆ alkyl, —NR₂R₂, and —COOR₃;
R₁₇ is selected from —H, —C₁₋₆ alkyl, —COOR₃, and aryl;
R₁₈ is selected from —COOR₂ and —C₁₋₆ alkyl-COOR₂;
R₁₉ is selected from —H, —C₁₋₆ alkyl, —C₁₋₆ alkyl-Q₄, —COR₃, —COOR₃,
wherein Q₄ is selected from —NR₂R₂ and —OR₂;
R₂₀ and R₂₁ are independently selected from —H, —C₁₋₆ alkyl, —C₁₋₆ substituted alkyl, —C₁₋₆ substituted alkyl-OR₂, and —COR₃,
or R₂₀ and R₂₁ are taken together with the adjacent N to form a cycle selected from

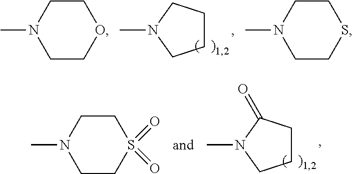

with the proviso that only one of R₂₀ or R₂₁ can be —COR₃;
R₂₂ and R₂₃ are independently selected from H, —C₁₋₆ alkyl, —C₁₋₆ substituted alkyl, and —C₁₋₆ cycloalkyl,
or R₂₂ and R₂₃ are taken together with the adjacent N to form a cycle selected from

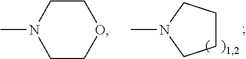

R₂₄ and R₂₅ are independently from the group of H, —C₁₋₆ alkyl, —C₁₋₆ substituted alkyl, —C₁₋₆ alkyl-Q₅, —C₁₋₆ cycloalkyl, aryl, substituted aryl, heteroaryl, and substituted heteroaryl, and Q₅ is selected from halogen and SO₂R₃,
R₂₆ and R₂₇ are independently selected from —H, —C₁₋₆ alkyl, —C₁₋₆ substituted alkyl, aryl, heteroaryl, substituted aryl, substituted heteroaryl, —C₁₋₆ alkyl-Q₂,
or R₂₆ and R₂₇ are taken together with the adjacent N to form a cycle selected from:

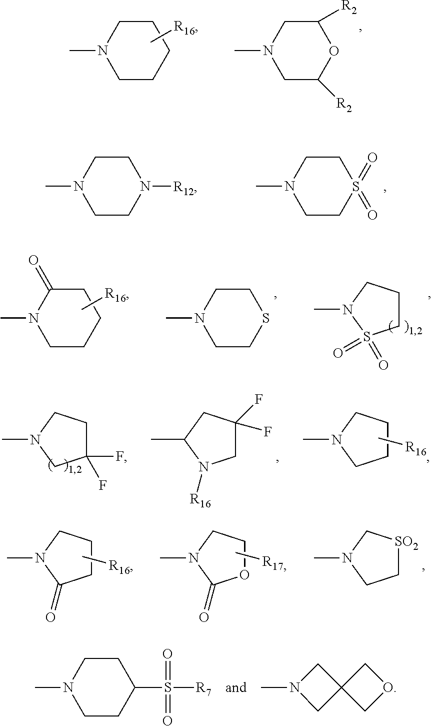

In an aspect of the invention, there is provided a compound of Formula I, wherein R₁ is isopropyl.

In an aspect of the invention, there is provided a compound of Formula, wherein X is phenyl.

In an aspect of the invention, there is provided a compound of Formula I, wherein Y is —COOR₂.

In an aspect of the invention, there is provided a compound of Formula I, wherein Y is —COOH.

In an aspect of the invention, there is provided a compound of Formula I, wherein A is —H.

In an aspect of the invention, there is provided a compound of Formula I, wherein R₄ is selected from —H, —C₁₋₆ alkyl, —C₁₋₆ alkyl-Q₁, and —COR₆.

In an aspect of the invention, there is provided a compound of Formula I, wherein R₅ is —H.

In an aspect of the invention, there is provided a compound of Formula I, wherein R₄ is —C₁₋₆ alkyl-Q₁.

In an aspect of the invention, there is provided a compound of Formula I, wherein Q₁ is —NR₈R₉.

In an aspect of the invention, there is provided a compound of Formula I, wherein R₄ is —COR₆.

In an aspect of the invention, there is provided a compound of Formula I, wherein W is —CH₂OR₂.

In an aspect of the invention, there is provided a compound of Formula I, wherein W is —COOR₂.

In an aspect of the invention, there is provided a compound of Formula I, wherein W is —COOH.

In an aspect of the invention, there is provided a compound of Formula I, wherein W is —NR₄R₅.

In an aspect of the invention, there is provided a compound of Formula I, wherein W is —CONR₂₆R₂₇.

In an aspect of the invention, there is provided a compound of Formula I, wherein W is —CH₂NR₂₆R₂₇.

In an aspect of the invention, there is provided a compound of Formula I, wherein W is —NR₄COR₆.

In an aspect of the invention, there is provided a compound of Formula I, wherein W is —NR₄C(O)NR₄R₅.

In an aspect of the invention, there is provided a compound of Formula I, wherein W is —NR₄COOR₆.

In an aspect of the invention, there is provided a compound, including pharmaceutically acceptable salts thereof, which is selected from:

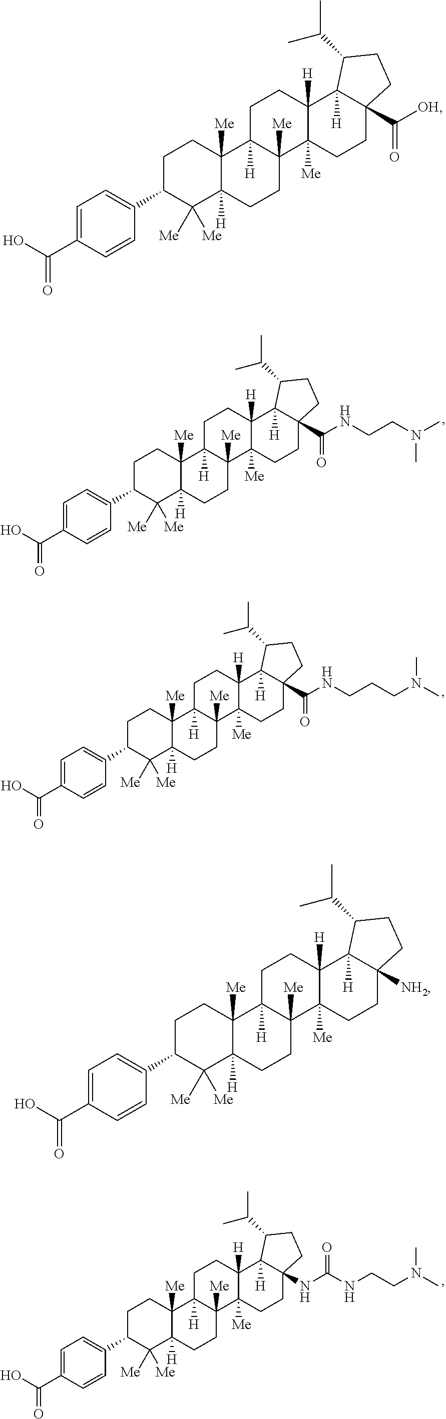

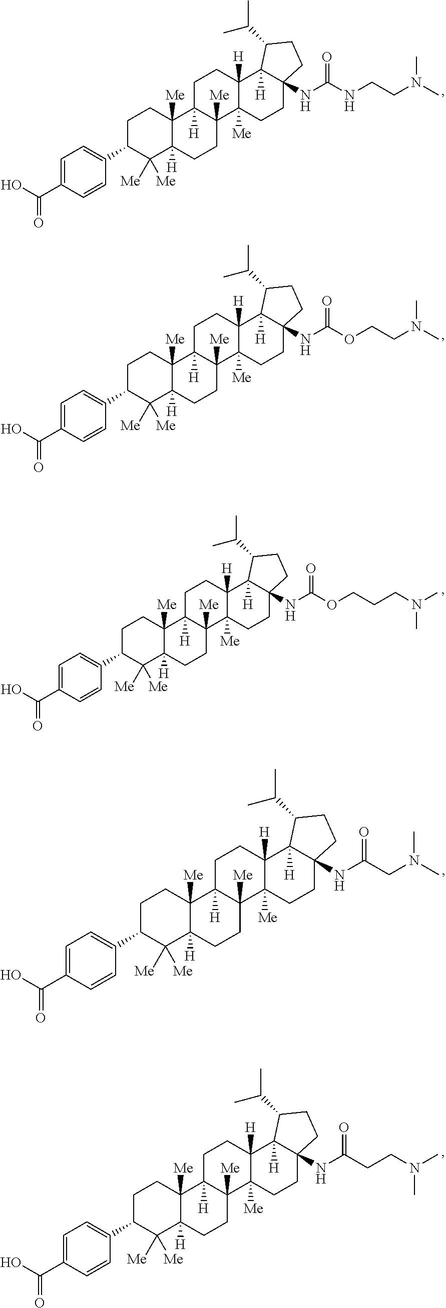

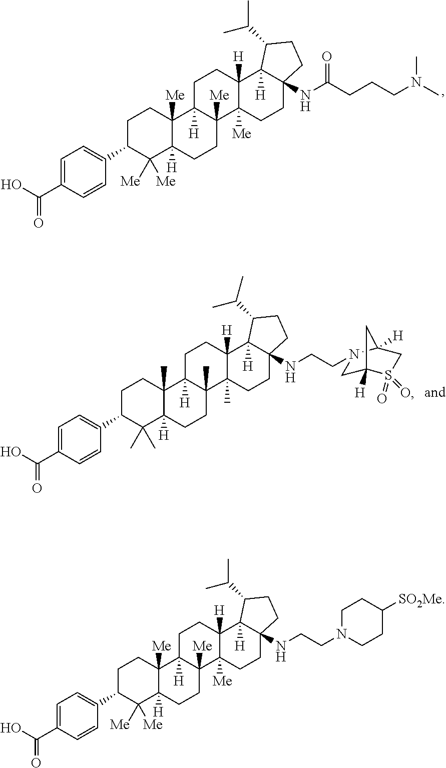

In an aspect of the invention, there is provided a pharmaceutical composition which comprises an antiviral effective amount of one or more of the compounds of the present invention, together with one or more pharmaceutically acceptable carriers, excipients or diluents.

In an aspect of the invention, there is provided a composition useful for treating HIV infection comprising a therapeutic amount of a compound of Formula I and a pharmaceutically acceptable carrier. In an aspect of the invention, the composition further comprises a therapeutically effective amount at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors, and a pharmaceutically acceptable carrier. In an aspect of the invention, the other agent is dolutegravir.

In an aspect of the invention, there is provided a method for treating HIV infection comprising administering a therapeutically effective amount of a compound of Formula I, or a pharmaceutically acceptable salt thereof, to a patient in need thereof. In an aspect of the invention, the method further comprises administering a therapeutically effective amount of at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors. In an aspect of the invention, the other agent is dolutegravir. In an aspect of the invention, the other agent is administered to the patient prior to, simultaneously with, or subsequently to the compound of Formula I.

### Pharmaceutical Compositions and Methods of Use

The compounds of the invention herein described and set forth are generally given as pharmaceutical compositions. These compositions are comprised of a therapeutically effective amount of a compound of Formula I or its pharmaceutically acceptable salt, and a pharmaceutically acceptable carrier and may contain conventional excipients and/or diluents. A therapeutically effective amount is that which is needed to provide a meaningful patient benefit. Pharmaceutically acceptable carriers are those conventionally known carriers having acceptable safety profiles. Compositions encompass all common solid and liquid forms, including capsules, tablets, lozenges, and powders, as well as liquid suspensions, syrups, elixirs, and solutions. Compositions are made using available formulation techniques, and excipients (such as binding and wetting agents) and vehicles (such as water and alcohols) which are generally used for compositions. See, for example, Remington's Pharmaceutical Sciences, 17th edition, Mack Publishing Company, Easton, Pa. (1985).

Solid compositions which are normally formulated in dosage units and compositions providing from about 1 to 1000 mg of the active ingredient per dose are preferred. Some examples of dosages are 1 mg, 10 mg, 100 mg, 250 mg, 500 mg, and 1000 mg. Generally, other antiretroviral agents will be present in a unit range similar to agents of that class used clinically. Typically, this is about 0.25-1000 mg/unit.

Liquid compositions are usually in dosage unit ranges. Generally, the liquid composition will be in a unit dosage range of about 1-100 mg/mL. Some examples of dosages are 1 mg/mL, 10 mg/mL, 25 mg/mL, 50 mg/mL, and 100 mg/mL. Generally, other antiretroviral agents will be present in a unit range similar to agents of that class used clinically. Typically, this is about 1-100 mg/mL.

The invention encompasses all conventional modes of administration; oral and parenteral methods are preferred. Generally, the dosing regimen will be similar to other antiretroviral agents used clinically. Typically, the daily dose will be about 1-100 mg/kg body weight daily. Generally, more compound is required orally and less parenterally. The specific dosing regimen, however, will be determined by a physician using sound medical judgment.

The compounds of this invention desirably have activity against HIV. Accordingly, another aspect of the invention is a method for treating HIV infection in a human patient comprising administering a therapeutically effective amount of a compound of Formula I, including a pharmaceutically acceptable salt thereof, with a pharmaceutically acceptable carrier, excipient and/or diluent.

The invention also encompasses methods where the compound is given in combination therapy. That is, the compound can be used in conjunction with, but separately from, other agents useful in treating AIDS and HIV infection. The compound can also be used in combination therapy wherein the compound and one or more of the other agents are physically together in a fixed-dose combination (FDC). Some of these agents include HIV attachment inhibitors, CCR5 inhibitors, CXCR4 inhibitors, HIV cell fusion inhibitors, HIV integrase inhibitors, HIV nucleoside reverse transcriptase inhibitors, HIV non-nucleoside reverse transcriptase inhibitors, HIV protease inhibitors, budding and maturation inhibitors, immunomodulators, and anti-infectives. In these combination methods, the compound of Formula I will generally be given in a daily dose of about 1-100 mg/kg body weight daily in conjunction with other agents. The other agents generally will be given in the amounts used therapeutically. The specific dosing regimen, however, will be determined by a physician using sound medical judgment.

“Combination,” “coadministration,” “concurrent” and similar terms referring to the administration of a compound of Formula I with at least one anti-HIV agent mean that the components are part of a combination antiretroviral therapy or HAART as understood by practitioners in the field of AIDS and HIV infection.

Thus, as set forth above, contemplated herein are combinations of the compounds of Formula I, together with one or more agents useful in the treatment of AIDS. For example, the compounds of the invention may be effectively administered, whether at periods of pre-exposure and/or post-exposure, in combination with effective amounts of the AIDS antivirals, immunomodulators, anti-infectives, or vaccines, such as those in the following non-limiting table:

| | | |
|---|---|---|
| Drug Name | Manufacturer | Indication |

ANTIVIRALS

| | | |
|---|---|---|
| Rilpivirine | Tibotec | HIV infection, AIDS, ARC |
| | | (non-nucleoside |
| | | reverse transcriptase |
| | | inhibitor) |
| COMPLERA ® | Gilead | HIV infection, AIDS, |
| | | ARC; combination |
| | | with emtricitabine, rilpivirine, |
| | | and tenofovir disoproxil |
| | | fumarate |
| 097 | Hoechst/Bayer | HIV infection, |
| | | AIDS, ARC |
| | | (non-nucleoside |
| | | reverse transcriptase |
| | | (RT) |
| | | inhibitor) |
| Amprenavir | Glaxo Wellcome | HIV infection, |
| 141 W94 | | AIDS, ARC |
| GW 141 | | (protease inhibitor) |
| Abacavir (1592U89) | Glaxo Wellcome | HIV infection, |
| GW 1592 | | AIDS, ARC |
| | | (RT inhibitor) |
| Acemannan | Carrington Labs | ARC |
| | (Irving, TX) | |
| Acyclovir | Burroughs Wellcome | HIV infection, AIDS, |
| | | ARC |
| AD-439 | Tanox Biosystems | HIV infection, AIDS, |
| | | ARC |
| AD-519 | Tanox Biosystems | HIV infection, AIDS, |
| | | ARC |
| Adefovir dipivoxil | Gilead Sciences | HIV infection |
| AL-721 | Ethigen | ARC, PGL |
| | (Los Angeles, CA) | HIV positive, AIDS |
| Alpha Interferon | Glaxo Wellcome | Kaposi's sarcoma, |
| | | HIV in combination w/Retrovir |
| Ansamycin | Adria Laboratories | ARC |
| LM 427 | (Dublin, OH) | |
| | Erbamont | |
| | (Stamford, CT) | |
| Antibody which | Advanced Biotherapy | AIDS, ARC |
| Neutralizes pH | Concepts | |
| Labile alpha aberrant | (Rockville, MD) | |
| Interferon | | |
| AR177 | Aronex Pharm | HIV infection, AIDS, |
| | | ARC |
| Beta-fluoro-ddA | Nat'l Cancer Institute | AIDS-associated |
| | | diseases |
| CI-1012 | Warner-Lambert | HIV-1 infection |
| Cidofovir | Gilead Science | CMV retinitis, |
| | | herpes, papillomavirus |
| Curdlan sulfate | AJI Pharma USA | HIV infection |
| Cytomegalovirus | MedImmune | CMV retinitis |
| Immune globin | | |
| Cytovene | Syntex | Sight threatening |
| Ganciclovir | | CMV |
| | | peripheral CMV |
| | | retinitis |
| Darunavir | Tibotec-J & J | HIV infection, AIDS, ARC |
| | | (protease inhibitor) |
| Delaviridine | Pharmacia-Upjohn | HIV infection, |
| | | AIDS, ARC |
| | | (RT inhibitor) |
| Dextran Sulfate | Ueno Fine Chem. | AIDS, ARC, HIV |
| | Ind. Ltd. (Osaka, | positive |
| | Japan) | asymptomatic |
| ddC | Hoffman-La Roche | HIV infection, AIDS, |
| Dideoxycytidine | | ARC |
| ddI | Bristol-Myers Squibb | HIV infection, AIDS, |
| Dideoxyinosine | | ARC; combination |
| | | with AZT/d4T |
| DMP-450 | AVID | HIV infection, |
| | (Camden, NJ) | AIDS, ARC |
| | | (protease inhibitor) |
| Efavirenz | Bristol Myers Squibb | HIV infection, |
| (DMP 266, SUSTIVA ®) | | AIDS, ARC |
| (−)6-Chloro-4-(S)- | | (non-nucleoside RT |
| cyclopropylethynyl- | | inhibitor) |
| 4(S)-trifluoro- | | |
| methyl-1,4-dihydro- | | |
| 2H-3,1-benzoxazin- | | |
| 2-one, STOCRINE | | |
| EL10 | Elan Corp, PLC | HIV infection |
| | (Gainesville, GA) | |
| Etravirine | Tibotec/J & J | HIV infection, AIDS, ARC |
| | | (non-nucleoside |
| | | reverse transcriptase |
| | | inhibitor) |
| Famciclovir | Smith Kline | herpes zoster, |
| | | herpes simplex |
| GS 840 | Gilead | HIV infection, |
| | | AIDS, ARC |
| | | (reverse transcriptase |
| | | inhibitor) |
| HBY097 | Hoechst Marion | HIV infection, |
| | Roussel | AIDS, ARC |
| | | (non-nucleoside |
| | | reverse transcriptase |
| | | inhibitor) |
| Hypericin | VIMRx Pharm. | HIV infection, AIDS, |
| | | ARC |
| Recombinant Human | Triton Biosciences | AIDS, Kaposi's |
| Interferon Beta | (Almeda, CA) | sarcoma, ARC |
| Interferon alfa-n3 | Interferon Sciences | ARC, AIDS |
| Indinavir | Merck | HIV infection, AIDS, |
| | | ARC, asymptomatic |
| | | HIV positive, also in |
| | | combination with |
| | | AZT/ddI/ddC |
| ISIS 2922 | ISIS Pharmaceuticals | CMV retinitis |
| KNI-272 | Nat'l Cancer Institute | HIV-assoc. diseases |
| Lamivudine, 3TC | Glaxo Wellcome | HIV infection, |
| | | AIDS, ARC |
| | | (reverse |
| | | transcriptase |
| | | inhibitor); also |
| | | with AZT |
| Lobucavir | Bristol-Myers Squibb | CMV infection |
| Nelfinavir | Agouron | HIV infection, |
| | Pharmaceuticals | AIDS, ARC |
| | | (protease inhibitor) |
| Nevirapine | Boeheringer | HIV infection, |
| | Ingleheim | AIDS, ARC |
| | | (RT inhibitor) |
| Novapren | Novaferon Labs, Inc. | HIV inhibitor |
| | (Akron, OH) | |
| Peptide T | Peninsula Labs | AIDS |
| Octapeptide | (Belmont, CA) | |
| Sequence | | |
| Trisodium | Astra Pharm. | CMV retinitis, HIV |
| Phosphonoformate | Products, Inc. | infection, other CMV |
| | | infections |
| PNU-140690 | Pharmacia Upjohn | HIV infection, |
| | | AIDS, ARC |
| | | (protease inhibitor) |
| Probucol | Vyrex | HIV infection, AIDS |
| RBC-CD4 | Sheffield Med. | HIV infection, |
| | Tech (Houston, TX) | AIDS, ARC |
| Ritonavir | Abbott | HIV infection, |
| | | AIDS, ARC |
| | | (protease inhibitor) |
| Saquinavir | Hoffmann- | HIV infection, |
| | LaRoche | AIDS, ARC |
| | | (protease inhibitor) |
| Stavudine; d4T | Bristol-Myers Squibb | HIV infection, AIDS, |
| Didehydrodeoxy- | | ARC |
| Thymidine | | |
| Tipranavir | Boehringer Ingelheim | HIV infection, AIDS, ARC |
| | | (protease inhibitor) |
| Valaciclovir | Glaxo Wellcome | Genital HSV & CMV |
| | | Infections |
| Virazole | Viratek/ICN | asymptomatic HIV |
| Ribavirin | (Costa Mesa, CA) | positive, LAS, ARC |
| VX-478 | Vertex | HIV infection, AIDS, |
| | | ARC |
| Zalcitabine | Hoffmann-LaRoche | HIV infection, AIDS, |
| | | ARC, with AZT |
| Zidovudine; AZT | Glaxo Wellcome | HIV infection, AIDS, |
| | | ARC, Kaposi's |
| | | sarcoma, in combination with |
| | | other therapies |
| Tenofovir disoproxil, | Gilead | HIV infection, |
| fumarate salt (VIREAD ®) | | AIDS, |
| | | (reverse transcriptase |
| | | inhibitor) |
| EMTRIVA ® | Gilead | HIV infection, |
| (Emtricitabine) (FTC) | | AIDS, |
| | | (reverse transcriptase |
| | | inhibitor) |
| COMBIVIR ® | GSK | HIV infection, |
| | | AIDS, |
| | | (reverse transcriptase |
| | | inhibitor) |
| Abacavir succinate | GSK | HIV infection, |
| (or ZIAGEN ®) | | AIDS, |
| | | (reverse transcriptase |
| | | inhibitor) |
| REYATAZ ® | Bristol-Myers Squibb | HIV infection |
| (or atazanavir) | | AIDs, protease |
| | | inhibitor |
| FUZEON ® | Roche/Trimeris | HIV infection |
| (Enfuvirtide or T-20) | | AIDs, viral Fusion |
| | | inhibitor |
| LEXIVA ® | GSK/Vertex | HIV infection |
| (or Fosamprenavir calcium) | | AIDs, viral protease |
| | | inhibitor |
| SELZENTRY ™ | Pfizer | HIV infection |
| Maraviroc; (UK 427857) | | AIDs, (CCR5 antagonist, in |
| | | development) |
| TRIZIVIR ® | GSK | HIV infection |
| | | AIDs, (three drug combination) |
| Sch-417690 (vicriviroc) | Schering-Plough | HIV infection |
| | | AIDs, (CCR5 antagonist, in |
| | | development) |
| TAK-652 | Takeda | HIV infection |
| | | AIDs, (CCR5 antagonist, in |
| | | development) |
| GSK 873140 | GSK/ONO | HIV infection |
| (ONO-4128) | | AIDs, (CCR5 antagonist, |
| | | in development) |
| Integrase Inhibitor | Merck | HIV infection |
| MK-0518 | | AIDs |
| Raltegravir | | |
| TRUVADA ® | Gilead | Combination of Tenofovir |
| | | disoproxil fumarate salt |
| | | (VIREAD ®) and EMTRIVA ® |
| | | (Emtricitabine) |
| Integrase Inhibitor | Gilead/Japan Tobacco | HIV Infection |
| GS917/JTK-303 | | AIDs |
| Elvitegravir | | in development |
| Triple drug combination | Gilead/Bristol-Myers Squibb | Combination of Tenofovir |
| ATRIPLA ® | | disoproxil fumarate salt |
| | | (VIREAD ®), EMTRIVA ® |
| | | (Emtricitabine), and |
| | | SUSTIVA ® (Efavirenz) |
| FESTINAVIR ® | Oncolys BioPharma | HIV infection |
| | | AIDs |
| | | in development |
| CMX-157 | Chimerix | HIV infection |
| Lipid conjugate of | | AIDs |
| nucleotide tenofovir | | |
| GSK1349572 | GSK | HIV infection |
| Integrase inhibitor | | AIDs |
| TIVICAY ® | | |
| dolutegravir | | |

IMMUNOMODULATORS

| | | |
|---|---|---|
| AS-101 | Wyeth-Ayerst | AIDS |
| Bropirimine | Pharmacia Upjohn | Advanced AIDS |
| Acemannan | Carrington Labs, Inc. | AIDS, ARC |
| | (Irving, TX) | |
| CL246,738 | Wyeth | AIDS, Kaposi's |
| | Lederle Labs | sarcoma |
| FP-21399 | Fuki ImmunoPharm | Blocks HIV fusion |
| | | with CD4+ cells |
| Gamma Interferon | Genentech | ARC, in combination |
| | | w/TNF (tumor |
| | | necrosis factor) |
| Granulocyte | Genetics Institute | AIDS |
| Macrophage Colony | Sandoz | |
| Stimulating Factor | | |
| Granulocyte | Hoechst-Roussel | AIDS |
| Macrophage Colony | Immunex | |
| Stimulating Factor | | |
| Granulocyte | Schering-Plough | AIDS, |
| Macrophage Colony | | combination |
| Stimulating Factor | | w/AZT |
| HIV Core Particle | Rorer | Seropositive HIV |
| Immunostimulant | | |
| IL-2 | Cetus | AIDS, in combination |
| Interleukin-2 | | w/AZT |
| IL-2 | Hoffman-LaRoche | AIDS, ARC, HIV, in |
| Interleukin-2 | Immunex | combination w/AZT |
| IL-2 | Chiron | AIDS, increase in |
| Interleukin-2 | | CD4 cell counts |
| (aldeslukin) | | |
| Immune Globulin | Cutter Biological | Pediatric AIDS, in |
| Intravenous | (Berkeley, CA) | combination w/AZT |
| (human) | | |
| IMREG-1 | Imreg | AIDS, Kaposi's |
| | (New Orleans, LA) | sarcoma, ARC, PGL |
| IMREG-2 | Imreg | AIDS, Kaposi's |
| | (New Orleans, LA) | sarcoma, ARC, PGL |
| Imuthiol Diethyl | Merieux Institute | AIDS, ARC |
| Dithio Carbamate | | |
| Alpha-2 | Schering Plough | Kaposi's sarcoma |
| Interferon | | w/AZT, AIDS |
| Methionine- | TNI Pharmaceutical | AIDS, ARC |
| Enkephalin | (Chicago, IL) | |
| MTP-PE | Ciba-Geigy Corp. | Kaposi's sarcoma |
| Muramyl-Tripeptide | | |
| Granulocyte | Amgen | AIDS, in combination |
| Colony Stimulating | | w/AZT |
| Factor | | |
| Remune | Immune Response | Immunotherapeutic |
| | Corp. | |
| rCD4 | Genentech | AIDS, ARC |
| Recombinant | | |
| Soluble Human CD4 | | |
| rCD4-IgG | | AIDS, ARC |
| hybrids | | |
| Recombinant | Biogen | AIDS, ARC |
| Soluble Human CD4 | | |
| Interferon | Hoffman-La Roche | Kaposi's sarcoma |
| Alfa 2a | | AIDS, ARC, |
| | | in combination w/AZT |
| SK&F106528 | Smith Kline | HIV infection |
| Soluble T4 | | |
| Thymopentin | Immunobiology | HIV infection |
| | Research Institute | |
| | (Annandale, NJ) | |
| Tumor Necrosis | Genentech | ARC, in combination |
| Factor; TNF | | w/gamma Interferon |

ANTI-INFECTIVES

| | | |
|---|---|---|
| Clindamycin with | Pharmacia Upjohn | PCP |
| Primaquine | | |
| Fluconazole | Pfizer | Cryptococcal |
| | | meningitis, |
| | | candidiasis |
| Pastille | Squibb Corp. | Prevention of |
| Nystatin Pastille | | oral candidiasis |
| Ornidyl | Merrell Dow | PCP |
| Eflornithine | | |
| Pentamidine | LyphoMed | PCP treatment |
| Isethionate (IM & IV) | (Rosemont, IL) | |
| Trimethoprim | | Antibacterial |
| Trimethoprim/sulfa | | Antibacterial |
| Piritrexim | Burroughs Wellcome | PCP treatment |
| Pentamidine | Fisons Corporation | PCP prophylaxis |
| Isethionate for | | |
| Inhalation | | |
| Spiramycin | Rhone-Poulenc | Cryptosporidial |
| | diarrhea | |
| Intraconazole- | Janssen-Pharm. | Histoplasmosis; |
| R51211 | | cryptococcal |
| | | meningitis |
| Trimetrexate | Warner-Lambert | PCP |
| Daunorubicin | NeXstar, Sequus | Kaposi's sarcoma |
| Recombinant Human | Ortho Pharm. Corp. | Severe anemia |
| Erythropoietin | | assoc. with AZT |
| | | therapy |
| Recombinant Human | Serono | AIDS-related |
| Growth Hormone | | wasting, cachexia |
| Megestrol Acetate | Bristol-Myers Squibb | Treatment of |
| | | anorexia assoc. |
| | | W/AIDS |
| Testosterone | Alza, Smith Kline | AIDS-related wasting |
| Total Enteral | Norwich Eaton | Diarrhea and |
| Nutrition | Pharmaceuticals | malabsorption |
| | | related to AIDS |

“Therapeutically effective” means the amount of agent required to provide a meaningful patient benefit as understood by practitioners in the field of AIDS and HIV infection. In general, the goals of therapeutically effective treatment include suppression of viral load, restoration and preservation of immunologic function, improved quality of life, and reduction of HIV-related morbidity and mortality.

“Patient” means a person infected with the HIV virus and suitable for therapy as understood by practitioners in the field of AIDS and HIV infection.

“Treatment,” “therapy,” “regimen,” “HIV infection,” “ARC,” “AIDS” and related terms are used as understood by practitioners in the field of AIDS and HIV infection.

### Methods of Synthesis

The compounds of the invention according to the various aspects can be made by various methods available in the art, including those of the following schemes in the specific examples which follow. The structure numbering and variable numbering shown in the synthetic schemes may be distinct from, and should not be confused with, the structure or variable numbering in the claims or the rest of the specification. The variables in the schemes are meant only to illustrate how to make some of the compounds of the invention.

Abbreviations used in the schemes generally follow conventions used in the art.

Some specific chemical abbreviations used in the examples are defined as follows: “DMF” for N,N-dimethylformamide; “MeOH” for methanol; “Ar” for aryl; “TFA” for trifluoroacetic acid; “BOC” for t-butoxycarbonate, “DMSO” for dimethylsulfoxide; “h” for hours; “rt” for room temperature or retention time (context will dictate); “min” for minutes; “EtOAc” for ethyl acetate; “THF” for tetrahydrofuran; “Et₂O” for diethyl ether; “DMAP” for 4-dimethylaminopyridine; “DCE” for 1,2-dichloroethane; “ACN” for acetonitrile; “DME” for 1,2-dimethoxyethane; “HATU” for (1-[Bis(dimethylamino)methylene]-1H-1,2,3-triazolo[4,5-b]pyridinium 3-oxid hexafluorophosphate) “DIEA” for diisopropylethylamine.

Certain other abbreviations as used herein, are defined as follows: “1×” for once, “2×” for twice, “3×” for thrice, “° C.” for degrees Celsius, “eq” for equivalent or equivalents, “g” for gram or grams, “mg” for milligram or milligrams, “L” for liter or liters, “mL” for milliliter or milliliters, “μL” for microliter or microliters, “N” for normal, “M” for molar, “mmol” for millimole or millimoles, “min” for minute or minutes, “h” for hour or hours, “rt” for room temperature, “RT” for retention time, “atm” for atmosphere, “psi” for pounds per square inch, “conc.” for concentrate, “sat” or “sat'd” for saturated, “MW” for molecular weight, “mp” for melting point, “ee” for enantiomeric excess, “MS” or “Mass Spec” for mass spectrometry, “ESI” for electrospray ionization mass spectroscopy, “HR” for high resolution, “HRMS” for high resolution mass spectrometry, “LCMS” for liquid chromatography mass spectrometry, “HPLC” for high pressure liquid chromatography, “RP HPLC” for reverse phase HPLC, “TLC” or “tlc” for thin layer chromatography, “NMR” for nuclear magnetic resonance spectroscopy, “¹H” for proton, “δ” for delta, “s” for singlet, “d” for doublet, “t” for triplet, “q” for quartet, “m” for multiplet, “br” for broad, “Hz” for hertz, and “α”, “β”, “R”, “S”, “E”, and “Z” are stereochemical designations familiar to one skilled in the art.

### EXAMPLES

The following examples illustrate typical syntheses of the compounds of Formula I, as described generally above. These examples are illustrative only and are not intended to limit the disclosure in any way. The reagents and starting materials are readily available to one of ordinary skill in the art.

### Chemistry

Typical Procedures and Characterization of Selected Examples: Unless otherwise stated, solvents and reagents were used directly as obtained from commercial sources, and reactions were performed under a nitrogen atmosphere. Flash chromatography was conducted on Silica gel 60 (0.040-0.063 particle size; EM Science supply). ¹H NMR spectra were recorded on Bruker DRX-500f at 500 MHz (or Bruker AV 400 MHz, Bruker DPX-300B or Varian Gemini 300 at 300 MHz as stated). The chemical shifts were reported in ppm on the δ scale relative to δTMS=0. The following internal references were used for the residual protons in the following solvents: CDCl₃ (δ_{H} 7.26), CD₃OD (δ_{H} 3.30), Acetic-d4 (Acetic Acid d₄) (δ_{H} 11.6, 2.07), DMSO mix or DMSO-D6_CDCl₃ ((_{H} 2.50 and 8.25) (ratio 75%:25%), and DMSO-D6 (δ_{H} 2.50). Standard acronyms were employed to describe the multiplicity patterns: s (singlet), br. s (broad singlet), d (doublet), t (triplet), q (quartet), m (multiplet), b (broad), app (apparent). The coupling constant (J) is in Hertz. All Liquid Chromatography (LC) data were recorded on a Shimadzu LC-10AS liquid chromatograph using a SPD-10AV UV-Vis detector with Mass Spectrometry (MS) data determined using a Micromass Platform for LC in electrospray mode.

### LCMS Methods:

### Method 1:

Start % B=0, Final % B=100 over 2 minute gradient, hold 100% B for 2 minutes
Flow Rate=1 mL/min

### Solvent A=90% Water, 10% Methanol, 0.1% TFA

### Solvent B=10% Water, 90% Methanol, 0.1% TFA

### Column=Phenomenex Luna 2.0×30 mm C18, 3 um

### Method 2:

Start % B=0, Final % B=100 over 2 minute gradient, hold 100% B for 1 minute
Flow Rate=1 mL/min

### Solvent A=90% Water, 10% Methanol, 0.1% TFA

### Solvent B=10% Water, 90% Methanol, 0.1% TFA

### Column=Phenomenex Luna 2.0×30 mm C18, 3 um

### Method 3:

Start % B=0, Final % B=100 over 2 minute gradient, hold 100% B for 4 minutes
Flow Rate=1 mL/min

### Solvent A=90% Water, 10% Methanol, 0.1% TFA

### Solvent B=10% Water, 90% Methanol, 0.1% TFA

### Column=Phenomenex Luna 2.0×30 mm C18, 3 um

### Method 4:

Start % B=0, Final % B=100 over 4 minute gradient, hold 100% B for 1 minute
Flow Rate=1 mL/min

### Solvent A=90% Water, 10% Methanol, 0.1% TFA

### Solvent B=10% Water, 90% Methanol, 0.1% TFA

### Column=Phenomenex Luna 2.0×30 mm C18, 3 um

### Method 5:

Start % B=30, Final % B=100 over 4 min gradient
Flow Rate=0.8 ml/min

### Wavelength=220 nM

### Solvent A=90% Water, 10% Methanol, 0.1% TFA

### Solvent B=10% Water, 90% Methanol, 0.1% TFA

### Column=Phenomenex Luna 2.0×50 mm C18, 3 um

### Preparative Methods:

### Method 1:

### Instrument: Thar SFC Prep 350

### Preparative Column: IB (3×25 cm, 5 μm)

BPR pressure: 100 bars

### Temperature: 30° C.

Flow rate: 160 mL/min

### Mobile Phase: CO₂/MeOH:THF (3:1) (75/25)

### Detector Wavelength: 240 nm

Separation Program: Stack injection
Injection: 1.5 mL with cycle time 2 mins
sample preparation: 24 g in 680 mL MeOH:THF (1:2), 35.3 mg/mL

### Method 2:

### Instrument: Thar SFC Prep 350

### Preparative Column: IB (3×25 cm, 5 μm)

BPR pressure: 100 bars

### Temperature: 30° C.

Flow rate: 160 mL/min

### Mobile Phase: CO₂/MeOH:THF (3:1) (75/25)

### Detector Wavelength: 240 nm

Separation Program: Stack injection
Injection: 1.25 mL with cycle time 1.5 mins
sample preparation: 18.2 g in 500 mL MeOH:THF (1:2), 36.4 mg/mL

### Method 3:

Start % B=30, Final % B=100 over 12 min gradient, hold at 100% B for 4 min
Flow Rate=50 ml/min

### Wavelength=220

Solvent A=90% Water, 10% acetonitrile, 0.1% TFA
Solvent B=10% Water, 90% acetonitrile, 0.1% TFA

### Column=Waters Sunfire C18, 5 μm, 30×150 mm

### Method 4:

Isocratic 45% B elution until all material eluted (manual fraction collection)
Flow Rate=50 ml/min

### Wavelength=220

Solvent A=90% Water, 10% acetonitrile, 0.1% TFA
Solvent B=10% Water, 90% acetonitrile, 0.1% TFA

### Column=Waters Sunfire C18, 5 μm, 30×150 mm

### Preparation of Compounds

### Preparation of (1S,3aS,5aR,5bR,7aS,11aS,11bR,13aR,13bR)-1-isopropyl-9-(4-(methoxycarbonyl)phenyl)-5a,5b,8,8,11a-pentamethylicosahydro-1H-cyclopenta[a]chrysene-3a-carboxylic acid

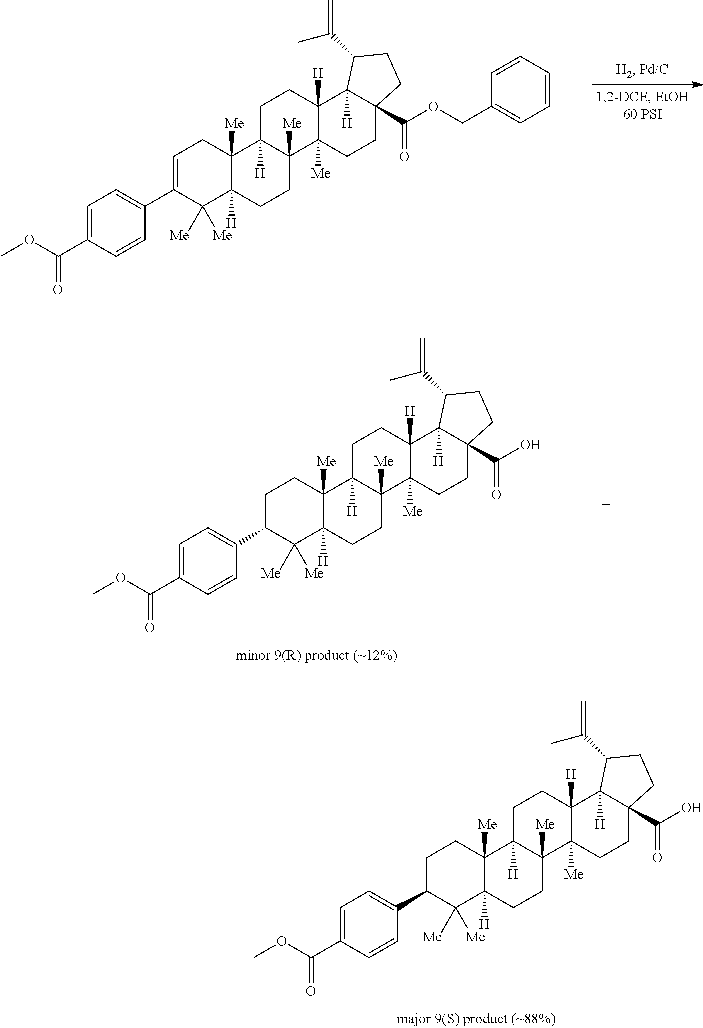

In a 2 L Parr hydrogenation vessel were combined (1R,3aS,5aR,5bR,7aR,11aS,11bR,13aR,13bR)-benzyl 9-(4-(methoxycarbonyl)phenyl)-5a,5b,8,8,11a-pentamethyl-1-(prop-1-en-2-yl)-2,3,3a,4,5,5a,5b,6,7,7a,8,11,11a,11b,12,13,13a,13b-octadecahydro-1H-cyclopenta[a]chrysene-3a-carboxylate (14.187 g, 21.40 mmol) and 1,2-DCE (120 mL). Then EtOH (120 mL) was added and the mixture was stirred and flushed with nitrogen. To the mixture was added 10% palladium on carbon (6.83 g, 6.42 mmol) in three separate portions. The vessel was charged with hydrogen gas on the Parr apparatus to 60 PSI and was shaken at rt. The reaction was removed from the Parr shaker after 64 h, and the reaction vessel was charged with more 10% palladium on carbon (3.42 g, 3.21 mmol) and 1,2-DCE (100 mL), EtOH (100 mL) and 1,4-dioxane (50 mL) were added and the vessel was recharged to 60 PSI with hydrogen gas on the Parr apparatus and shaken at rt for an additional 42 h. The catalyst was removed via filtration through a sintered glass funnel and the filtrate was concentrated in vacuo. The residue was dried in a vacuum oven at 50 degrees C. to give 12.47 g (quantitative yield) of a slightly yellow solid. This solid was a mixture of diastereomers (12% 9R isomer, 88% 9S isomer) formed during the hydrogenation. LCMS: m/z=577.4 (M+H)⁺, 4.19 min (Method 3). ¹H NMR (400 MHz, CHLOROFORM-d) δ ppm 7.90-7.97 (m, 2H), 7.29-7.32 (m, 0.25H), 7.25 (d, J=8.3 Hz, 1.75H), 3.92 (s, 3H), 2.81-2.88 (m, 0.12H), 2.42 (dd, J=13.2, 2.9 Hz, 0.88H), 2.22-2.34 (m, 3H), 2.11 (qd, J=13.4, 3.0 Hz, 1H), 1.92 (dd, J=12.5, 7.3 Hz, 1H), 1.78-1.88 (m, 2H), 1.69-1.76 (m, 1H), 1.57-1.68 (m, 3H), 1.47-1.57 (m, 4H), 1.39-1.47 (m, 5H), 1.29-1.39 (m, 3H), 1.17-1.25 (m, 2H), 1.03-1.11 (m, 1H), 1.02 (s, 3H), 1.00 (s, 3H), 0.98 (s, 3H), 0.94 (d, J=11.25 Hz, 1H), 0.89 (d, J=7 Hz, 3H), 0.80 (d, J=6.75 Hz, 3H), 0.78 (s, 3H), 0.72 (s, 2.6H), 0.55 (s, 0.4H).

### Purification of (1S,3aS,5aR,5bR,7aS,9R,11aS,11bR,13aR,13bR)-1-isopropyl-9-(4-(methoxycarbonyl)phenyl)-5a,5b,8,8,11a-pentamethylicosahydro-1H-cyclopenta[a]chrysene-3a-carboxylic acid

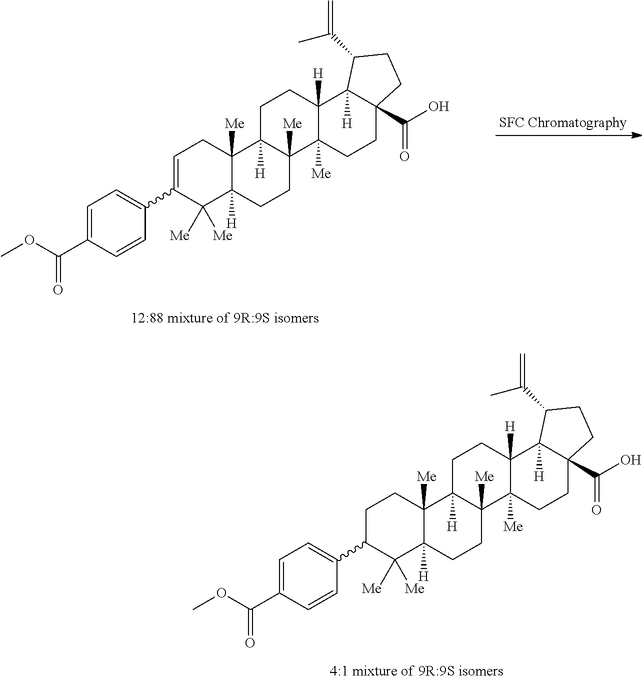

A 12:88 (9R:9S) mixture of diastereomers of (1S,3aS,5aR,5bR,7aS,11aS,11bR,13aR,13bR)-1-isopropyl-9-(4-(methoxycarbonyl)phenyl)-5a,5b,8,8,11a-pentamethylicosahydro-1H-cyclopenta[a]chrysene-3a-carboxylic acid (31.7 mmol) was purified via supercritical fluid chromatography in two passes (Preparative Method 1 and Preparative Method 2). Thus was isolated a 4:1 (9R:9S) mixture of diastereomers recovered as a white powder (2.0 g, 3.47 mmol, 10.9% yield). ¹H NMR (400 MHz, 1:1 mixture of CDCl3 and MeOD, MeOD lock) δ ppm 7.90 (d, J=8.3 Hz, 2H), 7.30 (d, J=8.1 Hz, 1.6H), 7.26 (d, J=8.1 Hz, 0.4H), 3.90 (s, 3H), 2.84 (dd, J=9.4, 2.8 Hz, 0.8H), 2.54-2.60 (m, 0.2H), 2.17-2.49 (m, 4H), 1.75-2.02 (m, 4H), 1.68 (d, J=11.7 Hz, 1H), 1.42-1.62 (m, 9H), 1.30-1.42 (m, 4H), 1.13-1.30 (m, 6H), 1.08 (s, 3H), 0.91-1.05 (m, 9H), 0.86 (d, J=6.6 Hz, 3H), 0.77 (d, J=6.8 Hz, 3H), 0.70 (s, 0.6H), 0.53 (s, 2.4H).

### Example B1

### Preparation of (1S,3aS,5aR,5bR,7aS,9R,11aS,11bR,13aR,13bR)-9-(4-carboxyphenyl)-1-isopropyl-5a,5b,8,8,8,11a-pentamethylicosahydro-1H-cyclopenta[a]chrysene-3a-carboxylic acid

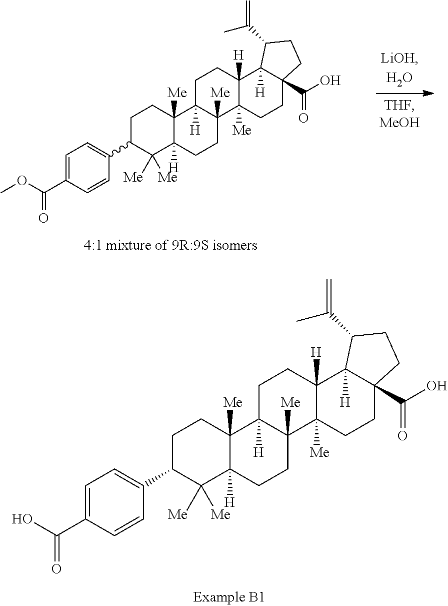

Solid (1S,3aS,5aR,5bR,7aS,11aS,11bR,13aR,13bR)-1-isopropyl-9-(4-(methoxycarbonyl)phenyl)-5a,5b,8,8,11a-pentamethylicosahydro-1H-cyclopenta[a]chrysene-3a-carboxylic acid as a 4:1 mixture of 9R:9S isomers (0.065 g, 0.113 mmol) was dissolved in tetrahydrofuran (0.90 mL) and MeOH (0.90 mL) and the resulting mixture was treated with lithium hydroxide hydrate (0.901 mL, 0.901 mmol). The mixture was heated to 75 degrees C. with stirring for 30 min. The crude mixture was purified by reverse phase preparative HPLC to provide the desired title 9R compound as the major product. The material was a neutral white powder (32.4 mg, 50.6% yield). LCMS: m/z=563.4 (M+H)⁺, 3.22 min (Method 1). ¹H NMR (400 MHz, Acetic) δ ppm 11.64 (s, 2H), 8.07-7.95 (m, J=8.1 Hz, 2H), 7.45-7.35 (m, J=8.1 Hz, 2H), 2.93 (dd, J=10.4, 2.3 Hz, 1H), 2.44-2.26 (m, 3H), 2.22 (br. s., 1H), 1.99-1.85 (m, 3H), 1.83 (br. s., 1H), 1.75 (d, J=11.5 Hz, 2H), 1.68-1.41 (m, 12H), 1.40-1.29 (m, 2H), 1.24 (d, J=13.0 Hz, 2H), 1.15 (s, 3H), 1.08 (s, 3H), 1.04 (s, 3H), 1.03 (s, 3H), 0.92 (d, J=6.6 Hz, 3H), 0.84 (d, J=6.8 Hz, 3H), 0.60 (s, 3H).

### Preparation of methyl 4-((1S,3aS,5aR,5bR,7aS,11aS,11bR,13aR,13bR)-3a-isocyanato-1-isopropyl-5a,5b,8,8,11a-pentamethylicosahydro-1H-cyclopenta[a]chrysen-9-yl)benzoate

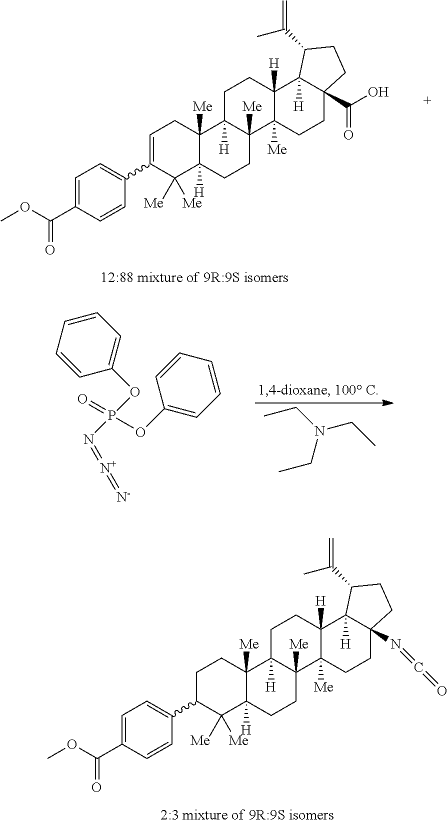

To a slurry of (1S,3aS,5aR,5bR,7aS,11aS,11bR,13aR,13bR)-1-isopropyl-9-(4-(methoxycarbonyl)phenyl)-5a,5b,8,8,11a-pentamethylicosahydro-1H-cyclopenta[a]chrysene-3a-carboxylic acid (12% 9(R) isomer, 88% 9(S) isomer) in 1,4-dioxane (250 mL) was added triethylamine (5.37 mL, 38.5 mmol) followed by diphenyl phosphoryl azide (7.13 mL, 32.1 mmol). The resulting yellow/orange slurry was heated to 100° C. which became a clear, orange solution. After 4.25 h the mixture was allowed to cool to rt and was concentrated in vacuo to a residue. The residue was taken up in chloroform (350 mL) and washed with water (2×200 mL) and then with a mixture of 1M NaOH (35 mL) and brine (50 mL). The hydroxide/brine wash was back-extracted with chloroform. The combined chloroform extracts were dried over MgSO₄, filtered and concentrated in vacuo until approximately 75 mL remained and a heavy precipitate of solid occurred. The resulting slurry was chilled in an ice bath and filtered, and the isolated solid was washed with ice cold ethyl acetate and allowed to air dry. The isolated solid (labeled isolate 01) was dried in a vacuum oven at 50 degrees C. to give 7.03 g (57.2% yield) as a white powder. This first isolate was highly enriched in the major 9(S) diastereomer and was set aside. The filtrate was concentrated in vacuo and the residue was purified by flash silica gel chromatography (220 g silica, elution gradient 100% hexanes to 20:1 hexanes:EtOAc). Distereomers were not separated and the chromatographed material (labeled isolate 02) was isolated as a 2.066 g of a white solid (16.8% yield). This second isolate was a mixture of isomers (40% 9(R) isomer, 60% 9(S) isomer). ¹H NMR (400 MHz, CHLOROFORM-d) δ ppm 7.95 (d, J=8.3 Hz, 0.8H), 7.94 (d, J=8.3 Hz, 1.2H), 7.30 (d, J=8.3 Hz, 0.8H), 7.26 (d, J=8.3 Hz, 1.2H), 3.92 (s, 3H), 2.85 (dd, J=9.7, 3.5 Hz, 0.4H), 2.42 (dd, J=13.3, 3.1 Hz, 0.6H), 2.05-2.18 (m, 0.6H), 1.76-1.94 (m, 8.4H), 1.66-1.73 (m, 1H), 1.38-1.59 (m, 10H), 1.15-1.37 (m, 6H), 1.10-1.13 (m, 4H), 0.99 (m, 6H), 0.91 (d, J=6.6 Hz, 4H), 0.76-0.81 (m, 5H), 0.73 (s, 1.8H), 0.55 (s, 1.2H).

### Example B2

### Preparation of 4-((1S,3aS,5aR,5bR,7aS,9R,11aS,11bR,13aR,13bR)-3a-((2-(dimethylamino)ethyl)carbamoyl)-1-isopropyl-5a,5b,8,8,11a-pentamethylicosahydro-1H-cyclopenta[a]chrysen-9-yl)benzoic acid

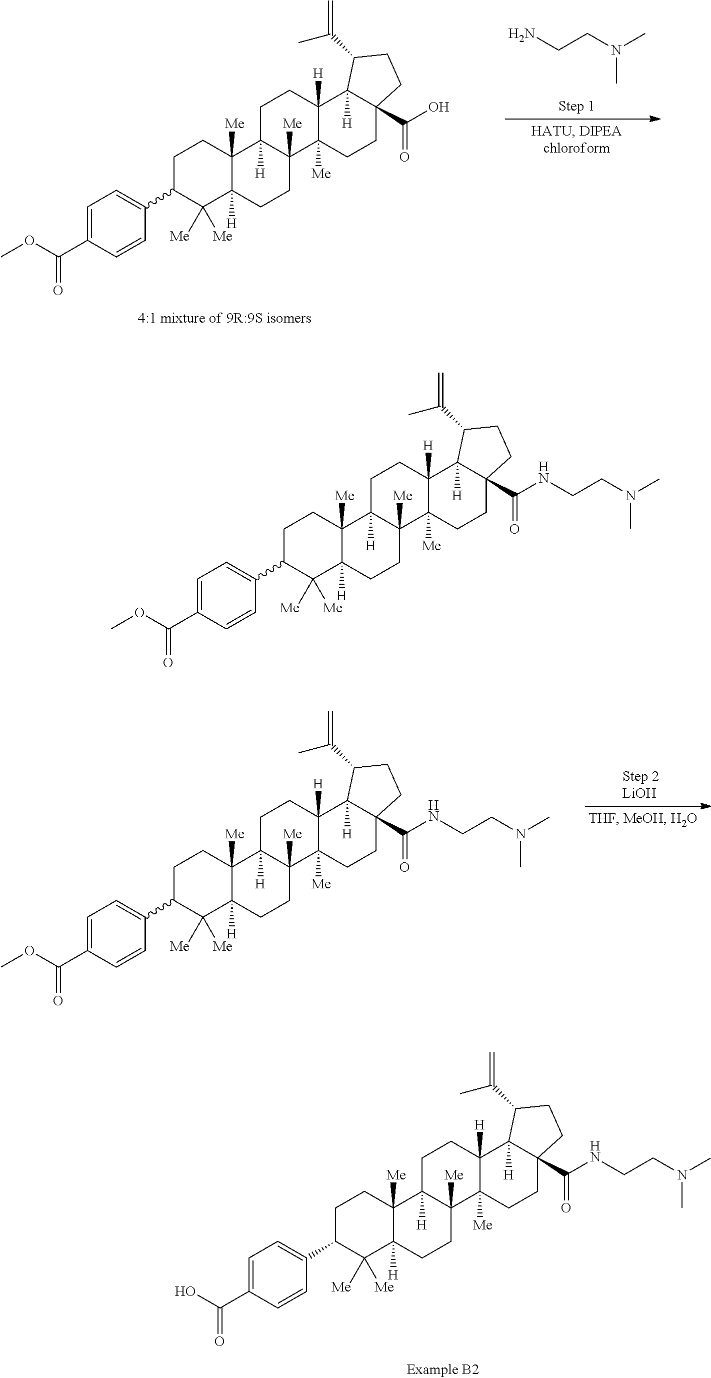

### Step 1. Preparation of methyl 4-((1S,3aS,5aR,5bR,7aS,11aS,11bR,13aR,13bR)-3a-((2-(dimethylamino)ethyl)carbamoyl)-1-isopropyl-5a,5b,8,8,11a-pentamethylicosahydro-1H-cyclopenta[a]chrysen-9-yl)benzoate

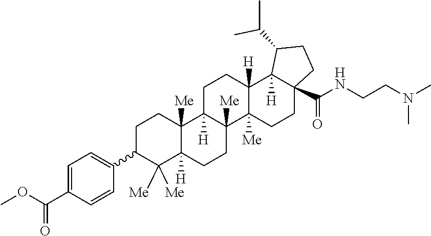

A 4:1 (9R:9S) mixture of isomers of (1S,3aS,5aR,5bR,7aS,11aS,11bR,13aR,13bR)-1-isopropyl-9-(4-(methoxycarbonyl)phenyl)-5a,5b,8,8,11a-pentamethylicosahydro-1H-cyclopenta[a]chrysene-3a-carboxylic acid (0.025 g, 0.043 mmol) was combined with HATU (0.021 g, 0.056 mmol) in chloroform (1 mL). To the stirred mixture was added N1,N1-dimethylethane-1,2-diamine (0.0050 g, 0.056 mmol) followed by DIPEA (0.017 g, 0.130 mmol). The mixture was stirred for 3 d and was then concentrated to a residue via nitrogen stream and was carried into the next step without purification. LCMS: m/z=647.5 (M+H)⁺, 2.63 min (Method 2).

### Step 2. Preparation of 4-((1S,3aS,5aR,5bR,7aS,9R,11aS,11bR,13aR,13bR)-3a-((2-(dimethylamino)ethyl)carbamoyl)-1-isopropyl-5a,5b,8,8,11a-pentamethylicosahydro-1H-cyclopenta[a]chrysen-9-yl)benzoic acid

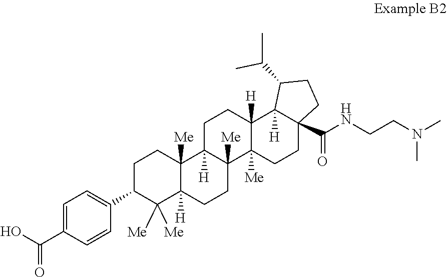

The residue from Step 1 was dissolved in a mixture of THF (0.3 mL) and MeOH (0.3 mL) and to the resulting solution was added 1.0M aqueous LiOH (0.34 mL, 0.34 mmol). The mixture was heated to 75° C. with stirring for 1.5 h. Purification by reverse phase preparative HPLC gave the major 9(R) isomer title compound (0.018 g, 55% yield) at a white powder TFA salt. LCMS: m/z=633.5 (M+H)⁺, 2.25 min (Method 1). ¹H NMR (400 MHz, 1:1 mixture of CDCl3 and MeOD, MeOD lock) δ ppm 7.91 (d, J=8.3 Hz, 2H), 7.29 (d, J=8.3 Hz, 2H), 3.51 (t, J=5.1 Hz, 2H), 3.19 (t, J=5.9 Hz, 2H), 2.92 (s, 6H), 2.84 (dd, J=9.5, 3.2 Hz, 1H), 2.43-2.53 (m, 1H), 2.21-2.31 (m, 1H), 2.07 (d, J=13.4 Hz, 1H), 1.59-1.93 (m, 6H), 1.43-1.58 (m, 10H), 1.31-1.43 (m, 4H), 1.17-1.31 (m, J=8.8 Hz, 4H), 1.08 (s, 3H), 1.03 (s, 3H), 0.97 (s, 3H), 0.96 (s, 3H), 0.86 (d, J=6.8 Hz, 3H), 0.77 (d, J=6.6 Hz, 3H), 0.54 (s, 3H).

### Example B3

### Preparation of 4-((1S,3aS,5aR,5bR,7aS,9R,11aS,11bR,13aR,13bR)-3a-((3-(dimethylamino)propyl)carbamoyl)-1-isopropyl-5a,5b,8,8,11a-pentamethylicosahydro-1H-cyclopenta[a]chrysen-9-yl)benzoic acid

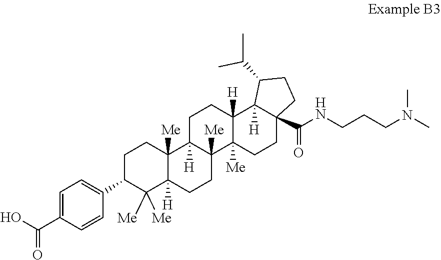

The title compound was prepared by the same procedure as described for the preparation of 4-((1S,3aS,5aR,5bR,7aS,9R,11aS,11bR,13aR,13bR)-3a-((2-(dimethylamino)ethyl)carbamoyl)-1-isopropyl-5a,5b,8,8,11a-pentamethylicosahydro-1H-cyclopenta[a]chrysen-9-yl)benzoic acid, except N1, N1-dimethylpropane-1,3-diamine (0.0058 g, 0.056 mmol) was used instead of N1,N1-dimethylethane-1,2-diamine in Step 1. The product was isolated as a white solid TFA salt (0.0125 g, 38% yield). LCMS: m/z=647.5 (M+H)⁺, 2.42 min (Method 1). ¹H NMR (400 MHz, 1:1 mixture of CDCl3 and MeOD, MeOD lock) δ ppm 7.91 (d, J=8.3 Hz, 2H), 7.68 (t, J=5.4 Hz, 1H), 7.29 (d, J=8.1 Hz, 2H), 3.20-3.30 (m, 2H), 3.01-3.11 (m, 2H), 2.88 (s, 6H), 2.84 (dd, J=9.5, 3.2 Hz, 1H), 2.42-2.54 (m, 1H), 2.28 (t, J=10.3 Hz, 1H), 2.08 (d, J=13.4 Hz, 1H), 1.64-1.98 (m, 7H), 1.43-1.61 (m, 9H), 1.32-1.42 (m, 5H), 1.15-1.32 (m, 5H), 1.08 (s, 3H), 1.04 (s, 3H), 0.97 (s, 3H), 0.96 (br. s., 3H), 0.86 (d, J=6.8 Hz, 3H), 0.77 (d, J=6.6 Hz, 3H), 0.54 (s, 3H).

### Example B4

### Preparation of 4-((1S,3aS,5aR,5bR,7aS,9R,11aS,11bR,13aR,13bR)-3a-amino-1-isopropyl-5a,5b,8,8,11a-pentamethylicosahydro-1H-cyclopenta[a]chrysen-9-yl)benzoic acid

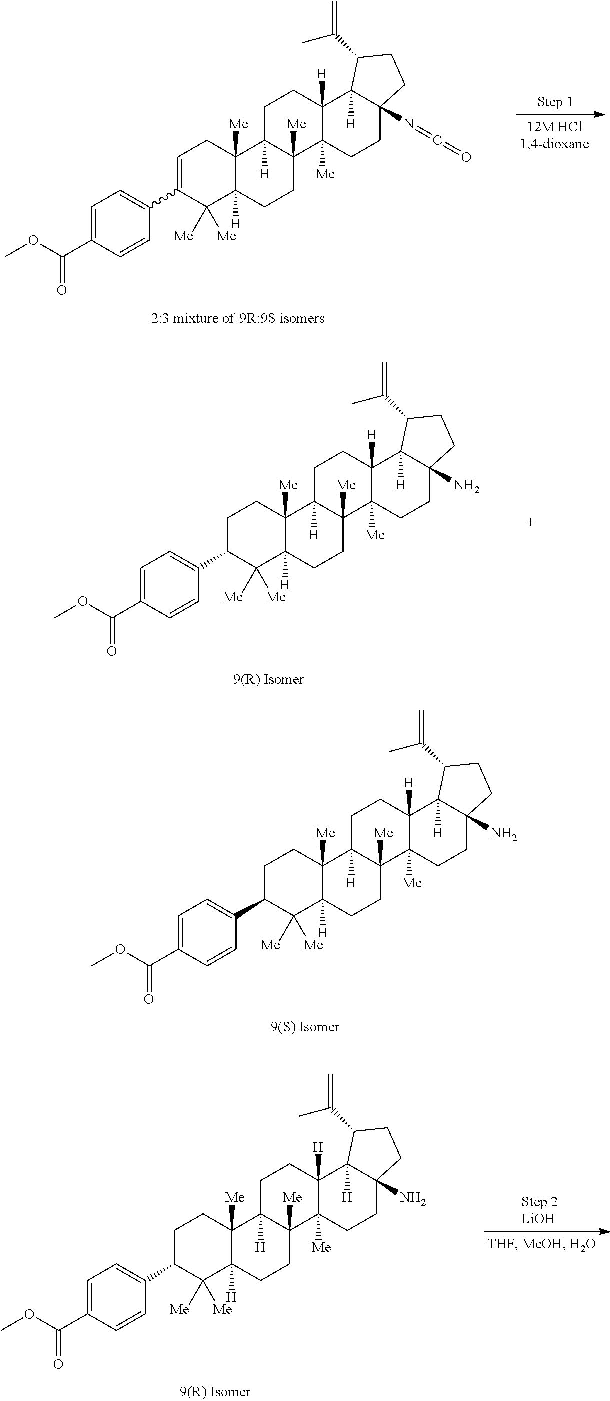

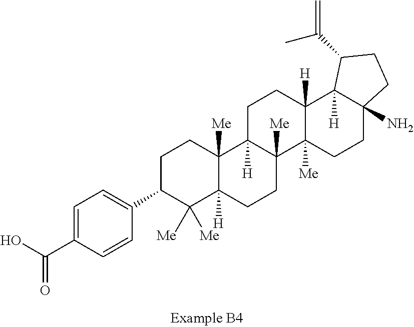

### Step 1. Preparation of methyl 4-((1S,3aS,5aR,5bR,7aS,9R,11aS,11bR,13aR,13bR)-3a-amino-1-isopropyl-5a,5b,8,8,11a-pentamethylicosahydro-1H-cyclopenta[a]chrysen-9-yl)benzoate

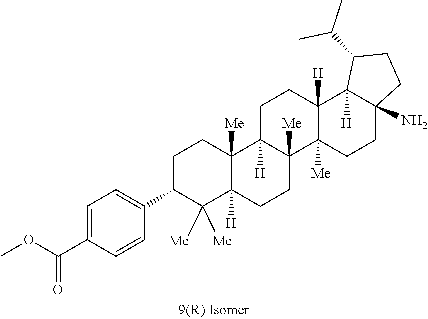

A 2:3 (9R:9S) mixture of isomers of methyl 4-((1S,3aS,5aR,5bR,7aS,11aS,11bR,13aR,13bR)-3a-isocyanato-1-isopropyl-5a,5b,8,8,11a-pentamethylicosahydro-1H-cyclopenta[a]chrysen-9-yl)benzoate (0.100 g, 0.174 mmol) was dissolved in 1,4-dioxane (1.5 mL) and the resulting solution was treated slowly with 12M HCl (0.145 mL, 1.73 mmol). The mixture was stirred at rt for 66 h and was then concentrated in vacuo to a white solid which was purified by reverse phase preparative HPLC to provide separation of the individual isomer compounds.

Isomer 1: methyl 4-((1S,3aS,5aR,5bR,7aS,9R,11aS,11bR,13aR,13bR)-3a-amino-1-isopropyl-5a,5b,8,8,11a-pentamethylicosahydro-1H-cyclopenta[a]chrysen-9-yl)benzoate. This isomer was the first to elute from the preparative HPLC. 37.9 mg white powder isolated as TFA salt (33.8% yield). LCMS: m/z=548.4 (M+H)⁺, 4.27 min (Method 4). ¹H NMR (400 MHz, 1:1 mixture of CDCl3 and MeOD, MeOD lock) δ ppm 7.91 (d, J=8.3 Hz, 2H), 7.30 (d, J=8.3 Hz, 2H), 3.90 (s, 3H), 2.85 (dd, J=9.5, 3.2 Hz, 1H), 1.97-2.04 (m, 1H), 1.82-1.96 (m, 4H), 1.62-1.82 (m, 8H), 1.53-1.61 (m, 3H), 1.39-1.53 (m, 8H), 1.22-1.37 (m, 4H), 1.13 (s, 3H), 1.11 (s, 3H), 1.06 (s, 3H), 0.97 (s, 3H), 0.92 (d, J=6.8 Hz, 3H), 0.83 (d, J=6.8 Hz, 3H), 0.55 (s, 3H).

Isomer 2: methyl 4-((1S,3aS,5aR,5bR,7aS,9S,11aS,11bR,13aR,13bR)-3a-amino-1-isopropyl-5a,5b,8,8,11a-pentamethylicosahydro-1H-cyclopenta[a]chrysen-9-yl)benzoate. This isomer was the second to elute from the preparative HPLC. 42.8 mg white powder isolated as TFA salt (38.1% yield). LCMS: m/z=548.4 (M+H)⁺, 4.27 min (Method 4). ¹H NMR (400 MHz, 1:1 mixture of CDCl3 and MeOD, MeOD lock) δ ppm 7.90 (d, J=8.3 Hz, 2H), 7.26 (d, J=8.3 Hz, 2H), 3.90 (s, 3H), 2.44 (dd, J=13.1, 3.1 Hz, 1H), 2.13 (qd, J=13.3, 3.3 Hz, 1H), 1.97-2.05 (m, 1H), 1.80-1.95 (m, 4H), 1.72-1.79 (m, 2H), 1.64-1.72 (m, 4H), 1.54-1.64 (m, 4H), 1.45 (d, J=13.7 Hz, 7H), 1.23-1.36 (m, 3H), 1.13 (s, 3H), 1.05 (s, 4H), 1.00 (s, 3H), 0.96 (d, J=11.5 Hz, 1H), 0.91 (d, J=6.8 Hz, 3H), 0.82 (d, J=6.6 Hz, 3H), 0.77 (s, 3H), 0.72 (s, 3H).

### Step 2. Preparation of 4-((1S,3aS,5aR,5bR,7aS,9R,11aS,11bR,13aR,13bR)-3a-amino-1-isopropyl-5a,5b,8,8,11a-pentamethylicosahydro-1H-cyclopenta[a]chrysen-9-yl)benzoic acid

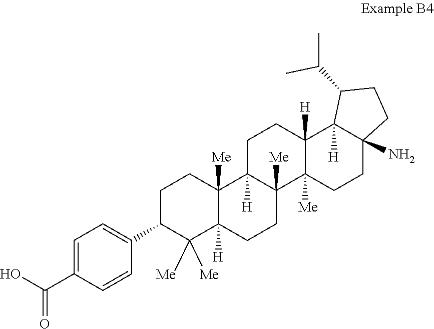

Methyl 4-((1S,3aS,5aR,5bR,7aS,9R,11aS,11bR,13aR,13bR)-3a-amino-1-isopropyl-5a,5b,8,8,11a-pentamethylicosahydro-1H-cyclopenta[a]chrysen-9-yl)benzoate TFA salt (0.031 g, 0.047 mmol) was combined with THF (0.3 mL), MeOH (0.3 mL) and 1M aqueous LiOH (0.23 mL, 0.23 mmol) and the resulting mixture was heated to 70 degrees C. for 45 min. Purification by reverse phase preparative HPLC gave the title compound as a white powder: 0.0207 g (67.5% yield) as the TFA salt. LCMS: m/z=534.4 (M+H)⁺, 2.32 min (Method 2). ¹H NMR (400 MHz, 1:1 mixture of CDCl3 and MeOD, MeOD lock) δ ppm 7.92 (d, J=8.3 Hz, 2H), 7.29 (d, J=8.3 Hz, 2H), 2.85 (dd, J=10.3, 2.7 Hz, 1H), 1.97-2.05 (m, 1H), 1.62-1.95 (m, 12H), 1.53-1.61 (m, 3H), 1.40-1.53 (m, 8H), 1.29-1.37 (m, 2H), 1.22-1.28 (m, 2H), 1.13 (s, 3H), 1.12 (s, 3H), 1.07 (s, 3H), 0.98 (s, 3H), 0.92 (d, J=6.8 Hz, 3H), 0.83 (d, J=6.6 Hz, 3H), 0.55 (s, 3H).

### Example B5

### Preparation of 4-((1S,3aS,5aR,5bR,7aS,9R,11aS,11bR,13aR,13bR)-3a-(3-(2-(dimethylamino)ethyl)ureido)-1-isopropyl-5a,5b,8,8,11a-pentamethylicosahydro-1H-cyclopenta[a]chrysen-9-yl)benzoic acid

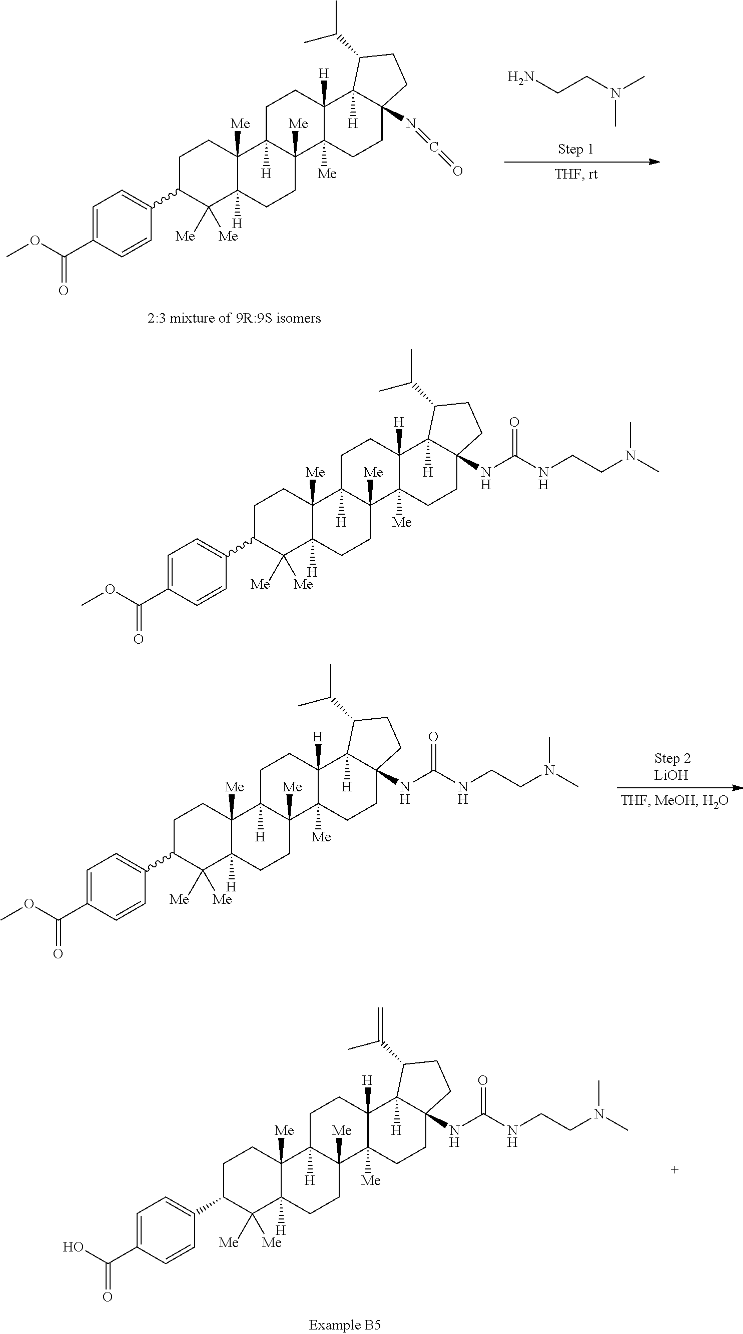

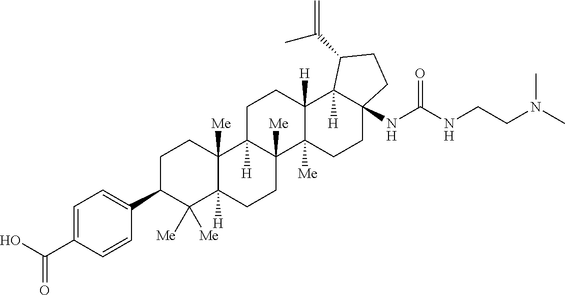

### Step 1: Preparation of methyl 4-((1S,3aS,5aR,5bR,7aS,11aS,11bR,13aR,13bR)-3a-(3-(2-(dimethylamino)ethyl)ureido)-1-isopropyl-5a,5b,8,8,11a-pentamethylicosahydro-1H-cyclopenta[a]chrysen-9-yl)benzoate

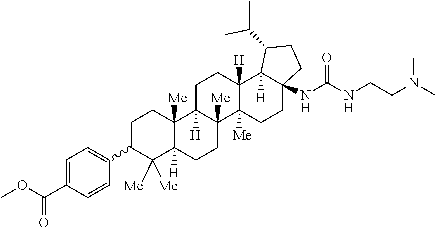

Under nitrogen atmosphere were combined the diastereomeric mixture methyl 4-((1S,3aS,5aR,5bR,7aS,11aS,11bR,13aR,13bR)-3a-isocyanato-1-isopropyl-5a,5b,8,8,11a-pentamethylicosahydro-1H-cyclopenta[a]chrysen-9-yl)benzoate (0.060 g, 0.105 mmol) with N1,N1-dimethylethane-1,2-diamine (0.023 ml, 0.209 mmol) in and anhydrous THF (1 mL). The mixture was stirred 1 h at rt and was then carried directly into the next step without purification. LCMS: m/z=662.6 (M+H)⁺, 2.63 min (Method 1).

### Step 2: Preparation of 4-((1S,3aS,5aR,5bR,7aS,9R,11aS,11bR,13aR,13bR)-3a-(3-(2-(dimethylamino)ethyl)ureido)-1-isopropyl-5a,5b,8,8,11a-pentamethylicosahydro-1H-cyclopenta[a]chrysen-9-yl)benzoic acid

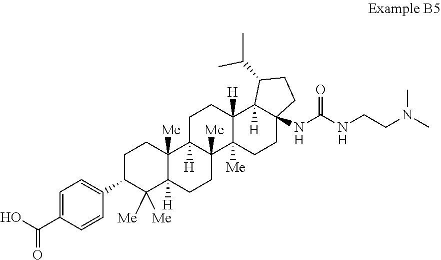

The crude mixture from Step 2 containing the diastereomeric mixture methyl 4-((1S,3aS,5aR,5bR,7aS,11aS,11bR,13aR,13bR)-3a-(3-(2-(dimethylamino)ethyl)ureido)-1-isopropyl-5a,5b,8,8,11a-pentamethylicosahydro-1H-cyclopenta[a]chrysen-9-yl)benzoate (0.070 g, 0.105 mmol) was diluted with MeOH (1 mL) and treated with 1M aqueous LiOH (0.84 mL, 0.840 mmol). The resulting mixture was heated to 75 degrees C. for 20 min with stirring. Purification of the crude mixture by reverse phase preparative HPLC provided separation of the diastereomeric title compounds.

This isomer was the first to elute from the preparative HPLC. 22.8 mg white powder isolated as TFA salt (27.6% yield). LCMS: m/z=648.5 (M+H)⁺, 2.42 min (Method 1). ¹H NMR (400 MHz, 1:1 mixture of CDCl3 and MeOD, MeOD lock) δ ppm 7.91 (d, J=8.1 Hz, 2H), 7.29 (d, J=8.3 Hz, 2H), 3.37-3.53 (m, 2H), 3.15-3.23 (m, 2H), 2.91 (s, 6H), 2.84 (dd, J=9.0, 3.2 Hz, 1H), 2.55 (d, J=13.4 Hz, 1H), 2.24 (dd, J=12.3, 7.0 Hz, 1H), 1.72-1.93 (m, 5H), 1.60-1.69 (m, 2H), 1.37-1.60 (m, 13H), 1.19-1.37 (m, 4H), 1.09 (s, 3H), 1.06 (s, 4H), 1.02 (s, 4H), 0.97 (s, 3H), 0.88 (d, J=6.8 Hz, 3H), 0.79 (d, J=6.6 Hz, 3H), 0.54 (s, 3H).

### Example B6

### Preparation of 4-((1S,3aS,5aR,5bR,7aS,9R,11aS,11bR,13aR,13bR)-3a-(3-(3-(dimethylamino)propyl)ureido)-1-isopropyl-5a,5b,8,8,11a-pentamethylicosahydro-1H-cyclopenta[a]chrysen-9-yl)benzoic acid

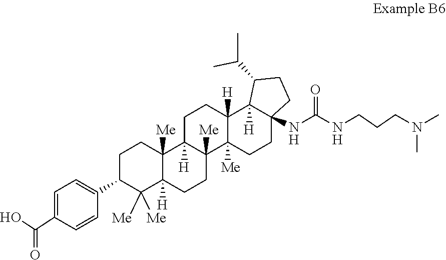

The title compound was prepared by the same procedure as described for the preparation of 4-((1S,3aS,5aR,5bR,7aS,9R,11aS,11bR,13aR,13bR)-3a-(((2-(dimethylamino)ethoxy)carbonyl)amino)-1-isopropyl-5a,5b,8,8,11a-pentamethylicosahydro-1H-cyclopenta[a]chrysen-9-yl)benzoic acid, except that N1,N1-dimethylpropane-1,3-diamine (0.026 ml, 0.209 mmol) was used instead of N1,N1-dimethylethane-1,2-diamine (0.023 ml, 0.209 mmol). Purification of the crude diastereomeric mixture by reverse phase preparative HPLC provided separation of the title compound.

This isomer was the first to elute from the preparative HPLC. 23.4 mg white powder isolated as TFA salt (28.4% yield). LCMS: m/z=662.5 (M+H)⁺, 2.41 min (Method 1). ¹H NMR (400 MHz, 1:1 mixture of CDCl3 and MeOD, MeOD lock) δ ppm 7.91 (d, J=8.3 Hz, 2H), 7.29 (d, J=8.3 Hz, 2H), 3.20-3.29 (m, 2H), 3.07 (t, J=6.7 Hz, 2H), 2.81-2.90 (m, 7H), 2.55 (d, J=13.2 Hz, 1H), 2.21 (dd, J=12.2, 6.6 Hz, 1H), 1.72-1.94 (m, 7H), 1.53-1.69 (m, 5H), 1.36-1.53 (m, 10H), 1.21-1.36 (m, 3H), 1.09 (s, 4H), 1.07 (s, 4H), 1.03 (s, 3H), 0.97 (s, 3H), 0.89 (d, J=6.6 Hz, 3H), 0.80 (d, J=6.6 Hz, 3H), 0.55 (s, 3H).

### Example B7

### Preparation of 4-((1S,3aS,5aR,5bR,7aS,9R,11aS,11bR,13aR,13bR)-3a-(((2-(dimethylamino)ethoxy)carbonyl)amino)-1-isopropyl-5a,5b,8,8,11a-pentamethylicosahydro-1H-cyclopenta[a]chrysen-9-yl)benzoic acid

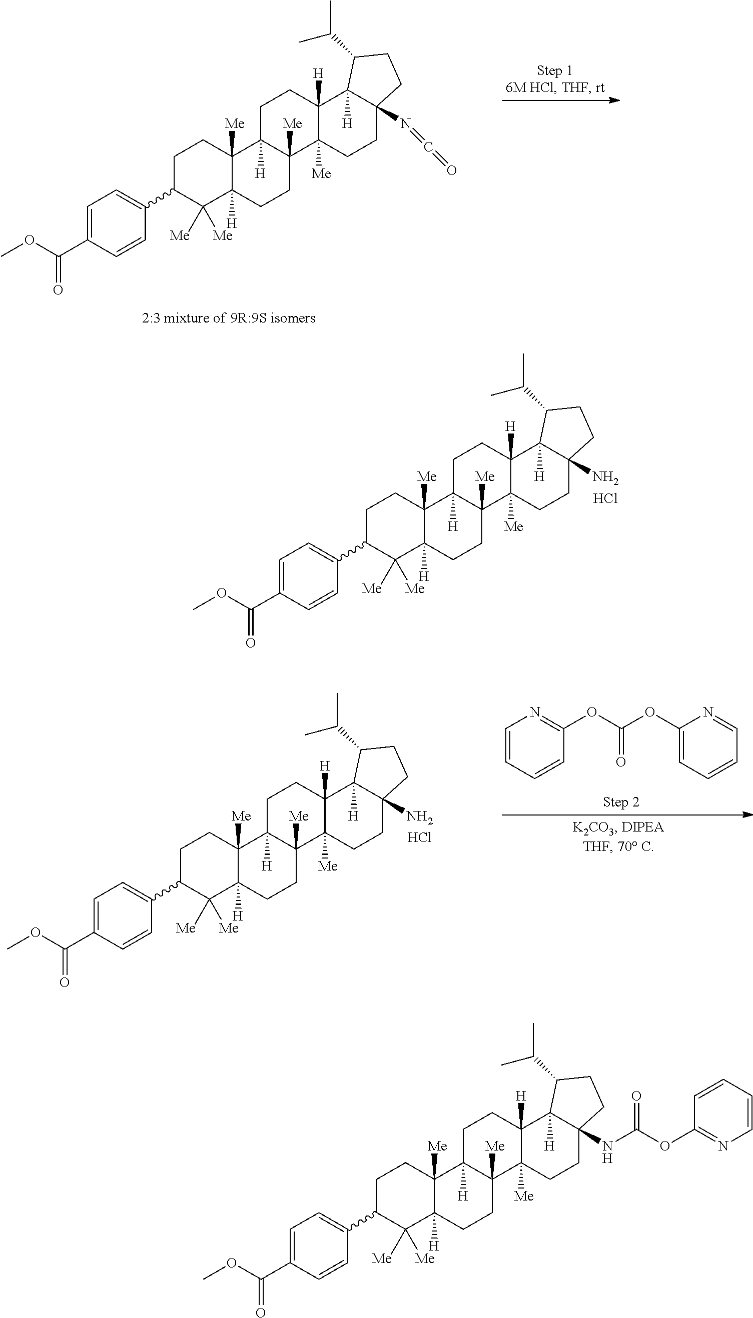

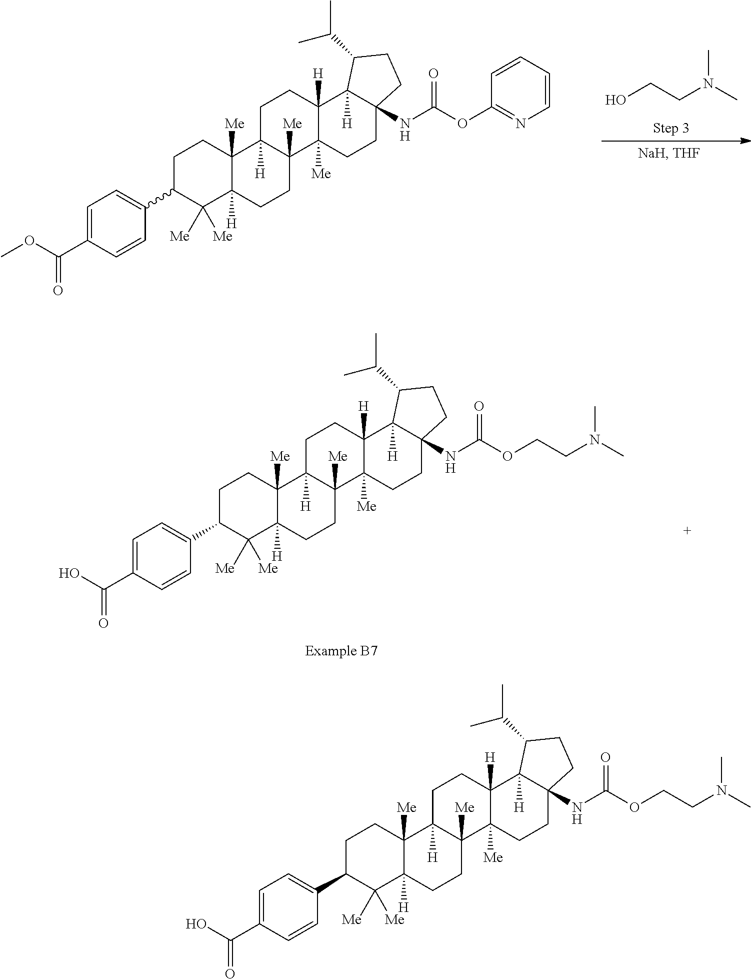

### Step 1: Preparation of methyl 4-((1S,3aS,5aR,5bR,7aS,11aS,11bR,13aR,13bR)-3a-amino-1-isopropyl-5a,5b,8,8,11a-pentamethylicosahydro-1H-cyclopenta[a]chrysen-9-yl)benzoate hydrochloride

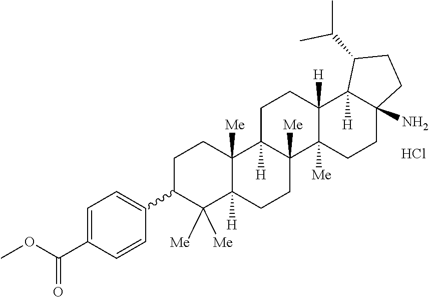

A 2:3 mixture of isomers of methyl 4-((1S,3aS,5aR,5bR,7aS,11aS,11bR,13aR,13bR)-3a-isocyanato-1-isopropyl-5a,5b,8,8,11a-pentamethylicosahydro-1H-cyclopenta[a]chrysen-9-yl)benzoate (0.85 g, 1.48 mmol) was dissolved in 1,4-dioxane (20 mL) and the resulting solution was treated slowly with 6M HCl (4.94 mL, 29.6 mmol). The mixture was stirred at rt for 66 h and was then concentrated in vacuo to afford the desired hydrochloride salt product mixture of isomers as a white solid: 0.866 g (quantitative). LCMS: m/z=548.5 (M+H)⁺, 2.47 min (Method 1).

### Step 2: Preparation of methyl 4-((1S,3aS,5aR,5bR,7aS,11aS,11bR,13aR,13bR)-1-isopropyl-5a,5b,8,8,11a-pentamethyl-3a-(((pyridin-2-yloxy)carbonyl)amino)icosahydro-1H-cyclopenta[a]chrysen-9-yl)benzoate

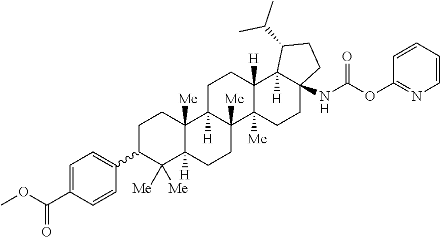

To a solution of the product from Step 1 (0.060 g; 0.103 mmol) in THF (1 mL) was added di(pyridin-2-yl) carbonate (0.024 g, 0.113 mmol), followed by addition of solid K₂CO₃ (0.028 g, 0.205 mmol) and DIPEA (0.027 g, 0.205 mmol). The resulting mixture was heated to 70° C. After 2 h, additional di(pyridin-2-yl) carbonate (0.024 g, 0.113 mmol) was added and heating to 70° C. was resumed for an additional 10 min. The crude mixture of isomers was carried directly into the next step.

### Step 3: Preparation of 4-((1S,3aS,5aR,5bR,7aS,9R,11aS,11bR,13aR,13bR)-3a-(((2-(dimethylamino)ethoxy)carbonyl)amino)-1-isopropyl-5a,5b,8,8,11a-pentamethylicosahydro-1H-cyclopenta[a]chrysen-9-yl)benzoic acid

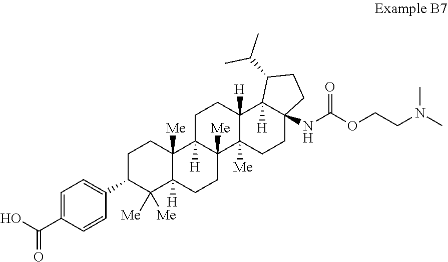

To a suspension of sodium hydride, 60% dispersion in oil (0.033 g, 0.824 mmol) in THF (1 mL) was slowly added 2-(dimethylamino)ethanol (0.073 g; 0.824 mmol). Vigorous bubbling immediately began upon addition and a slight exotherm resulted. The mixture was stirred at rt for 30 min and was then added all at once to the crude mixture from Step 2. The mixture was stirred for 90 min at rt. The methyl ester was hydrolized during the course of the reaction to provide product as the carboxylic acid. Purification by reverse phase preparative HPLC provided separation of the title compound.

This isomer was the first to elute from the preparative HPLC. 11.0 mg white powder isolated as TFA salt (13.9% yield). LCMS: m/z=649.6 (M+H)+, 2.47 min (Method 2). ¹H NMR (400 MHz, 1:1 mixture of CDCl3 and MeOD, MeOD lock) δ ppm 7.91 (d, J=8.1 Hz, 2H), 7.29 (d, J=8.1 Hz, 2H), 5.84 (s, 1H), 4.32 (tdd, J=18.2, 13.3, 4.9 Hz, 2H), 3.38 (t, J=4.8 Hz, 2H), 2.93 (s, 6H), 2.84 (dd, J=9.0, 3.2 Hz, 1H), 2.51 (d, J=12.7 Hz, 1H), 2.20 (dd, J=12.6, 6.7 Hz, 1H), 1.73-1.97 (m, 5H), 1.60-1.72 (m, 2H), 1.35-1.60 (m, 13H), 1.21-1.35 (m, 4H), 1.07-1.13 (m, 4H), 1.05 (s, 3H), 1.03 (s, 3H), 0.97 (s, 3H), 0.88 (d, J=6.6 Hz, 3H), 0.80 (d, J=6.6 Hz, 3H), 0.54 (s, 3H).

### Example B8

### Preparation of 4-((1S,3aS,5aR,5bR,7aS,9R,11aS,11bR,13aR,13bR)-3a-(((3-(dimethylamino)propoxy)carbonyl)amino)-1-isopropyl-5a,5b,8,8,11a-pentamethylicosahydro-1H-cyclopenta[a]chrysen-9-yl)benzoic acid

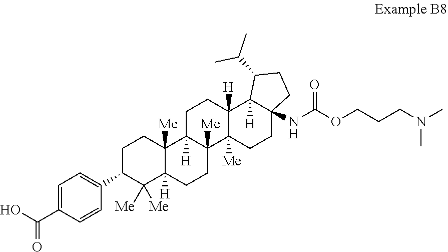

The title compound was prepared by a similar procedure as described for the preparation of 4-((1S,3aS,5aR,5bR,7aS,9R,11aS,11bR,13aR,13bR)-3a-(((2-(dimethylamino)ethoxy)carbonyl)amino)-1-isopropyl-5a,5b,8,8,11a-pentamethylicosahydro-1H-cyclopenta[a]chrysen-9-yl)benzoic acid, except that 3-(dimethylamino)propan-1-ol (0.085 g, 0.824 mmol) was used instead of 2-(dimethylamino)ethanol in Step 3. Purification of the crude diastereomeric mixture by reverse phase preparative HPLC provided separation of the title compound.

This isomer was the first to elute from the preparative HPLC. 19.7 mg white powder isolated as TFA salt (24.4% yield). LCMS: m/z=663.6 (M+H)⁺, 2.49 min (Method 2). ¹H NMR (400 MHz, 1:1 mixture of CDCl3 and MeOD, MeOD lock) δ ppm 7.91 (d, J=8.1 Hz, 2H), 7.29 (d, J=8.3 Hz, 2H), 5.55 (s, 1H), 4.10 (br. s., 2H), 3.13-3.23 (m, 2H), 2.89 (s, 6H), 2.84 (dd, J=9.4, 2.8 Hz, 1H), 2.51 (d, J=12.5 Hz, 1H), 2.21 (dd, J=11.6, 7.0 Hz, 1H), 2.05 (dt, J=12.9, 6.4 Hz, 2H), 1.71-1.95 (m, 5H), 1.67 (d, J=12.7 Hz, 1H), 1.35-1.63 (m, 14H), 1.18-1.35 (m, 3H), 1.09 (s, 4H), 1.06 (s, 4H), 1.03 (s, 3H), 0.97 (s, 3H), 0.89 (d, J=6.8 Hz, 3H), 0.80 (d, J=6.6 Hz, 3H), 0.55 (s, 3H).

### Example B9

### Preparation of 4-((1S,3aS,5aR,5bR,7aS,9R,11aS,11bR,13aR,13bR)-3a-(2-(dimethylamino)acetamido)-1-isopropyl-5a,5b,8,8,11a-pentamethylicosahydro-1H-cyclopenta[a]chrysen-9-yl)benzoic acid

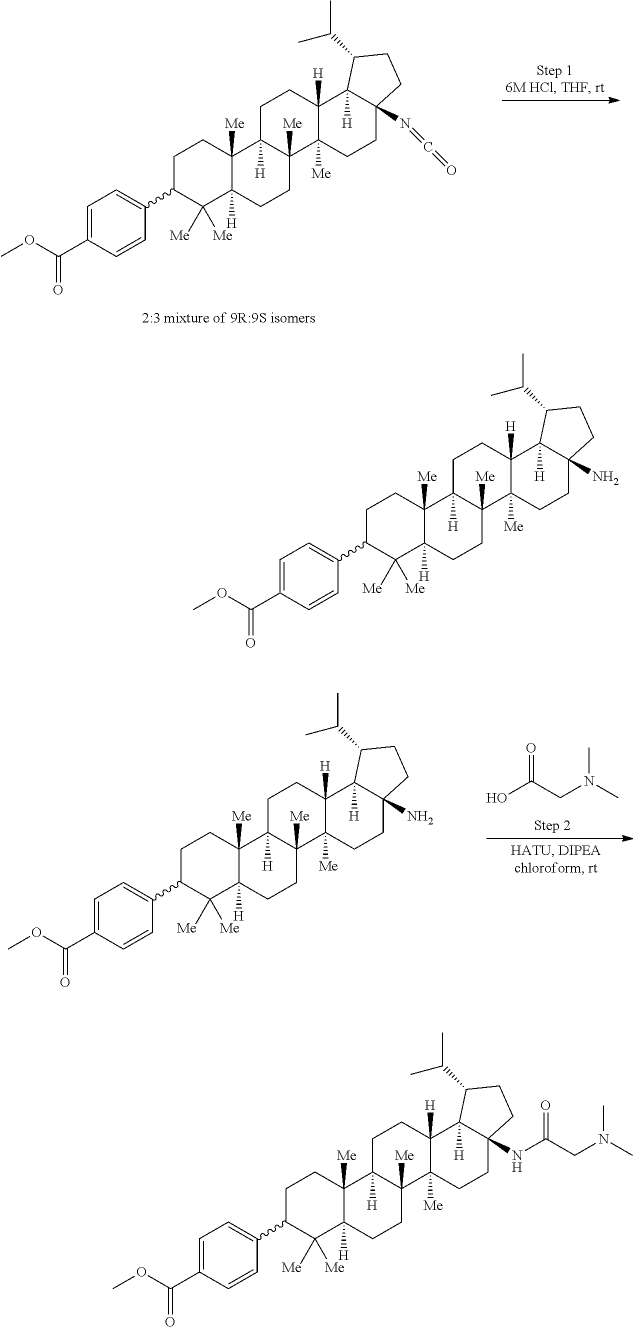

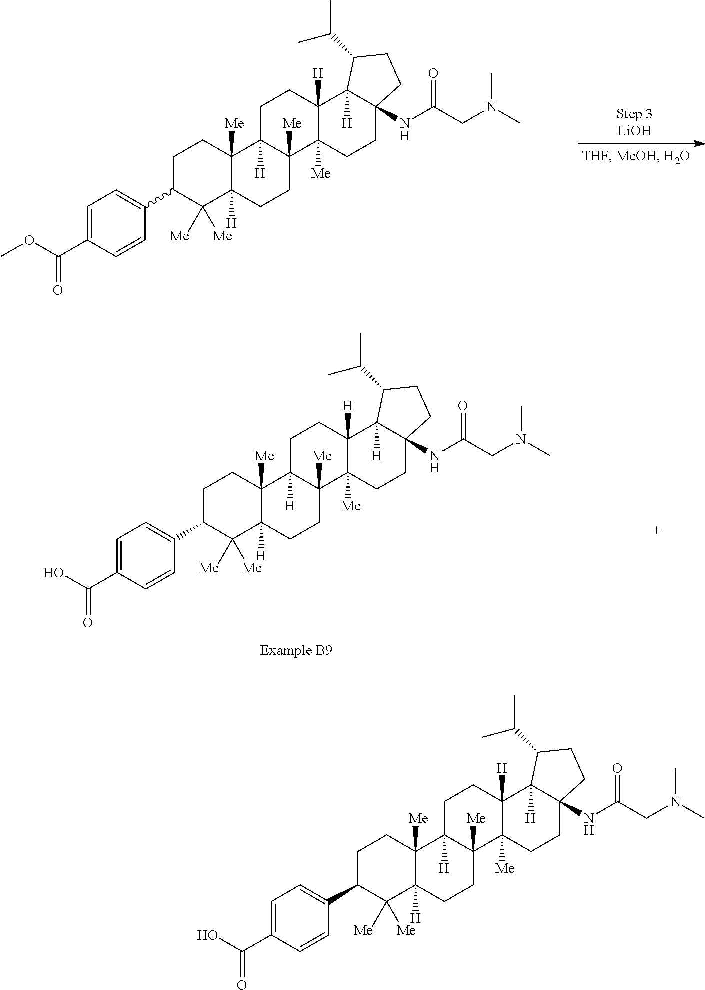

### Step 1: Preparation of methyl 4-((1S,3aS,5aR,5bR,7aS,11aS,11bR,13aR,13bR)-3a-amino-1-isopropyl-5a,5b,8,8,11a-pentamethylicosahydro-1H-cyclopenta[a]chrysen-9-yl)benzoate

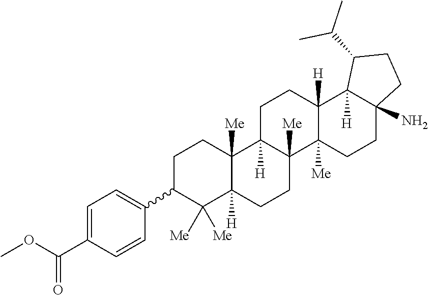

In a 20 mL scintillation vial were combined the diastereomeric mixture methyl 4-((1S,3aS,5aR,5bR,7aS,11aS,11bR,13aR,13bR)-3a-isocyanato-1-isopropyl-5a,5b,8,8,11a-pentamethylicosahydro-1H-cyclopenta[a]chrysen-9-yl)benzoate (0.75 g, 1.307 mmol) with 1,4-dioxane (10 mL). Then, HYDROCHLORIC ACID, 6M (2.178 mL, 13.07 mmol) was slowly added and the mixture was stirred at rt for 3 d. The mixture was partly concentrated under nitrogen stream and to the residue was added ethyl acetate (150 mL). This mixture was slowly treated with saturated aqueous sodium bicarbonate (50 mL) and the resulting mixture was shaken carefully and phases were separated. The organic phase was dried over anhydrous magnesium sulfate, filtered and concentrated in vacuo to a white solid. Purification by flash silica gel chromatography (80 g silica, elution gradient 100% DCM to 20:1 DCM:MeOH) gave the desired product as a white solid: 0.653 g (91% yield). This material was a diastereomeric mixture. LCMS: m/z=548.4 (M+H)⁺, 2.46 min (Method 2). ¹H NMR (400 MHz, CHLOROFORM-d) δ ppm 7.95 (d, J=8.2 Hz, 0.8H), 7.94 (d, J=8.2 Hz, 1.2H), 7.30 (d, J=8.6 Hz, 0.8H), 7.25 (d, J=8.1 Hz, 1.2H), 5.32 (s, 1H), 3.92 (s, 3H), 2.85 (dd, J=9.8, 3.2 Hz, 0.4H), 2.60 (d, J=13.2 Hz, 0.4H), 2.42 (dd, J=13.3, 2.8 Hz, 0.6H), 2.27 (dd, J=11.2, 7.6 Hz, 0.4H), 2.11 (qd, J=13.3, 3.3 Hz, 0.6H), 1.65-1.99 (m, 7.6H), 1.57-1.64 (m, 2H), 1.43-1.57 (m, 8H), 1.40 (br. s., 4H), 1.19-1.35 (m, 3H), 1.10 (s, 5H), 1.05 (s, 2H), 0.98 (d, J=2.7 Hz, 7H), 0.86-0.94 (m, 4H), 0.78 (s, 5H), 0.72 (s, 1.8H), 0.53-0.58 (m, 1.2H).

### Step 2: Preparation of methyl 4-((1S,3aS,5aR,5bR,7aS,11aS,11bR,13aR,13bR)-3a-(2-(dimethylamino)acetamido)-1-isopropyl-5a,5b,8,8,11a-pentamethylicosahydro-1H-cyclopenta[a]chrysen-9-yl)benzoate

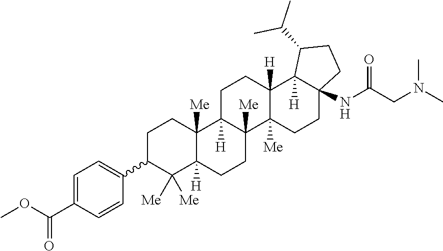

In a 1 dram vial were combined methyl 4-((1S,3aS,5aR,5bR,7aS,11aS,11bR,13aR,13bR)-3a-amino-1-isopropyl-5a,5b,8,8,8,11a-pentamethylicosahydro-1H-cyclopenta[a]chrysen-9-yl)benzoate (0.060 g, 0.110 mmol) with HATU (0.054 g, 0.142 mmol) and 2-(dimethylamino)acetic acid hydrochloride (0.020 g, 0.142 mmol) in chloroform (1 mL). To the stirred mixture was added DIPEA (0.077 mL, 0.438 mmol). The mixture was stirred at rt for 3 d. The mixture was concentrated via nitrogen stream and carried directly into the next step without purification. LCMS: m/z=633.5 (M+H)⁺, 2.60 min (Method 2).

### Step 3: Preparation of 4-((1S,3aS,5aR,5bR,7aS,9R,11aS,11bR,13aR,13bR)-3a-(2-(dimethylamino)acetamido)-1-isopropyl-5a,5b,8,8,11a-pentamethylicosahydro-1H-cyclopenta[a]chrysen-9-yl)benzoic acid

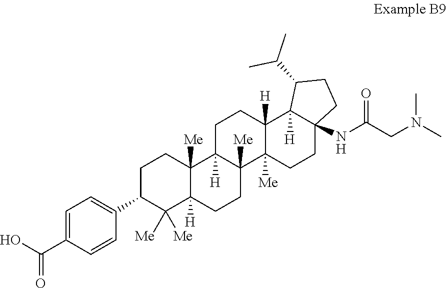

The crude diastereomeric mixture methyl 4-((1S,3aS,5aR,5bR,7aS,11aS,11bR,13aR,13bR)-3a-(2-(dimethylamino)acetamido)-1-isopropyl-5a,5b,8,8,11a-pentamethylicosahydro-1H-cyclopenta[a]chrysen-9-yl)benzoate from Step 2 was combined with THF (1 mL), methanol (1 mL) and 1.0M aqueous LiOH (0.88 mmol, 0.88 mL). The mixture was heated to 75 degrees C. for 2 h, then was treated with additional 1.0M aqueous LiOH (0.88 mmol, 0.88 mL) and heated to 80 degrees C. for an additional 90 min. The mixture was concentrated via nitrogen stream to a residue. Ethyl acetate (10 mL) and water (3 mL) were added and the mixture was shaken and phases were separated. The aqueous was extracted twice more with ethyl acetate (2×5 mL), the organic extracts were combined, concentrated in vacuo, and redissolved in THF to make prep HPLC samples. Purification of the crude diastereomeric mixture by reverse phase preparative HPLC provided separation of the title compound.

This isomer was the first to elute from the preparative HPLC. 13.2 mg white powder isolated as TFA salt (15.9% yield). LCMS: m/z=619.5 (M+H)⁺, 2.44 min (Method 1). ¹H NMR (400 MHz, 1:1 mixture of CDCl3 and MeOD, MeOD lock) δ ppm 7.92 (d, J=8.1 Hz, 2H), 7.36 (s, 1H), 7.29 (d, J=8.3 Hz, 2H), 3.80-3.93 (m, 2H), 2.90 (s, 6H), 2.84 (dd, J=3.2 Hz, 1H), 2.68 (d, J=13.4 Hz, 1H), 2.28 (d, J=12.5 Hz, 1H), 1.84-1.99 (m, 4H), 1.79 (br. s., 1H), 1.69 (d, J=11.5 Hz, 1H), 1.36-1.61 (m, 14H), 1.21-1.35 (m, 4H), 1.13 (br. s., 1H), 1.10 (s, 3H), 1.07 (s, 3H), 1.03 (s, 3H), 0.97 (s, 3H), 0.89 (d, J=6.6 Hz, 3H), 0.80 (d, J=6.4 Hz, 3H), 0.55 (s, 3H).

### Example B10

### Preparation of 4-((1S,3aS,5aR,5bR,7aS,9R,11aS,11bR,13aR,13bR)-3a-(3-(dimethylamino)propanamido)-1-isopropyl-5a,5b,8,8,11a-pentamethylicosahydro-1H-cyclopenta[a]chrysen-9-yl)benzoic acid

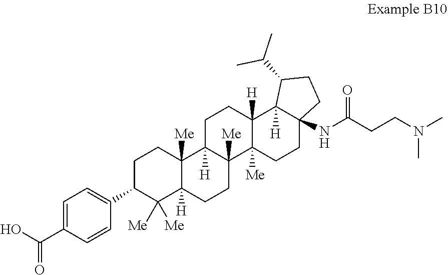

The title compound was prepared by the same procedure as described in the preparation of 4-((1S,3aS,5aR,5bR,7aS,9R,11aS,11bR,13aR,13bR)-3a-(2-(dimethylamino)acetamido)-1-isopropyl-5a,5b,8,8,11a-pentamethylicosahydro-1H-cyclopenta[a]chrysen-9-yl)benzoic acid, except 3-(dimethylamino)propanoic acid hydrochloride (0.022 g, 0.142 mmol) was used in place of 2-(dimethylamino)acetic acid hydrochloride in Step 2. Also, in Step 3, the crude product was not extracted from the reaction mixture but rather the reaction mixture was purified directly by reverse phase preparative HPLC to provide separation of the title compound.

This isomer was the first to elute from the preparative HPLC. 18.3 mg white powder isolated as TFA salt (21.6% yield). LCMS: m/z=633.5 (M+H)⁺, 2.45 min (Method 1). ¹H NMR (400 MHz, 1:1 mixture of CDCl3 and MeOD, MeOD lock) δ ppm 7.92 (d, J=8.1 Hz, 2H), 7.30 (d, J=8.1 Hz, 2H), 7.01 (s, 1H), 2.87 (s, 6H), 2.82-2.86 (m, 1H), 2.69-2.78 (m, 2H), 2.61-2.69 (m, 1H), 2.27 (dd, J=12.2, 5.1 Hz, 1H), 1.83-2.00 (m, 4H), 1.73-1.83 (m, 1H), 1.69 (d, J=10.8 Hz, 1H), 1.44-1.64 (m, 12H), 1.35-1.43 (m, 3H), 1.20-1.35 (m, 3H), 1.10 (s, 4H), 1.07 (s, 4H), 1.03 (s, 3H), 0.97 (s, 3H), 0.89 (d, J=6.6 Hz, 3H), 0.80 (d, J=6.6 Hz, 3H), 0.55 (s, 3H).

### Example B11

### Preparation of 4-((1S,3aS,5aR,5bR,7aS,9R,11aS,11bR,13aR,13bR)-3a-(4-(dimethylamino)butanamido)-1-isopropyl-5a,5b,8,8,11a-pentamethylicosahydro-1H-cyclopenta[a]chrysen-9-yl)benzoic acid

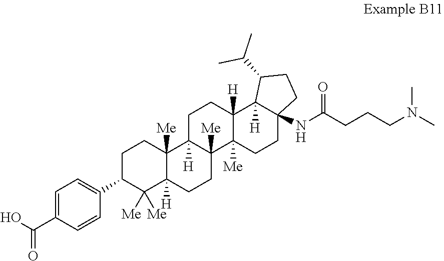

The title compound was prepared by the same procedure as described in the preparation of 4-((1S,3aS,5aR,5bR,7aS,9R,11aS,11bR,13aR,13bR)-3a-(2-(dimethylamino)acetamido)-1-isopropyl-5a,5b,8,8,11a-pentamethylicosahydro-1H-cyclopenta[a]chrysen-9-yl)benzoic acid, except 3-(dimethylamino)butanoic acid hydrochloride (0.024 g, 0.142 mmol) was used in place of 2-(dimethylamino)acetic acid hydrochloride in Step 2. Also, in Step 3, the crude product was not extracted from the reaction mixture but rather the reaction mixture was purified directly by reverse phase preparative HPLC to provide separation of the title compound.

This isomer was the first to elute from the preparative HPLC. 18.9 mg white powder isolated as TFA salt (21.9% yield). LCMS: m/z=647.5 (M+H)⁺, 2.45 min (Method 1). ¹H NMR (400 MHz, 1:1 mixture of CDCl3 and MeOD, MeOD lock) δ ppm 7.92 (d, J=8.3 Hz, 2H), 7.30 (d, J=8.3 Hz, 2H), 3.13 (t, J=7.1 Hz, 2H), 2.88 (s, 6H), 2.84 (dd, J=8.8, 3.2 Hz, 1H), 2.66 (d, J=13.4 Hz, 1H), 2.45 (t, J=6.6 Hz, 2H), 2.27 (d, J=11.5 Hz, 1H), 1.82-2.00 (m, 6H), 1.73-1.82 (m, 1H), 1.65-1.72 (m, 1H), 1.39-1.62 (m, 13H), 1.20-1.39 (m, 5H), 1.10 (s, 4H), 1.06 (s, 4H), 1.03 (s, 3H), 0.97 (s, 3H), 0.89 (d, J=6.6 Hz, 3H), 0.80 (d, J=6.6 Hz, 3H), 0.55 (s, 3H).

### Example B12

### Preparation of 4-((1S,3aS,5aR,5bR,7aS,9R,11aS,11bR,13aR,13bR)-3a-((2-((1S,4S)-2,2-dioxido-2-thia-5-azabicyclo[2.2.1]heptan-5-yl)ethyl)amino)-1-isopropyl-5a,5b,8,8,11a-pentamethylicosahydro-1H-cyclopenta[a]chrysen-9-yl)benzoic acid

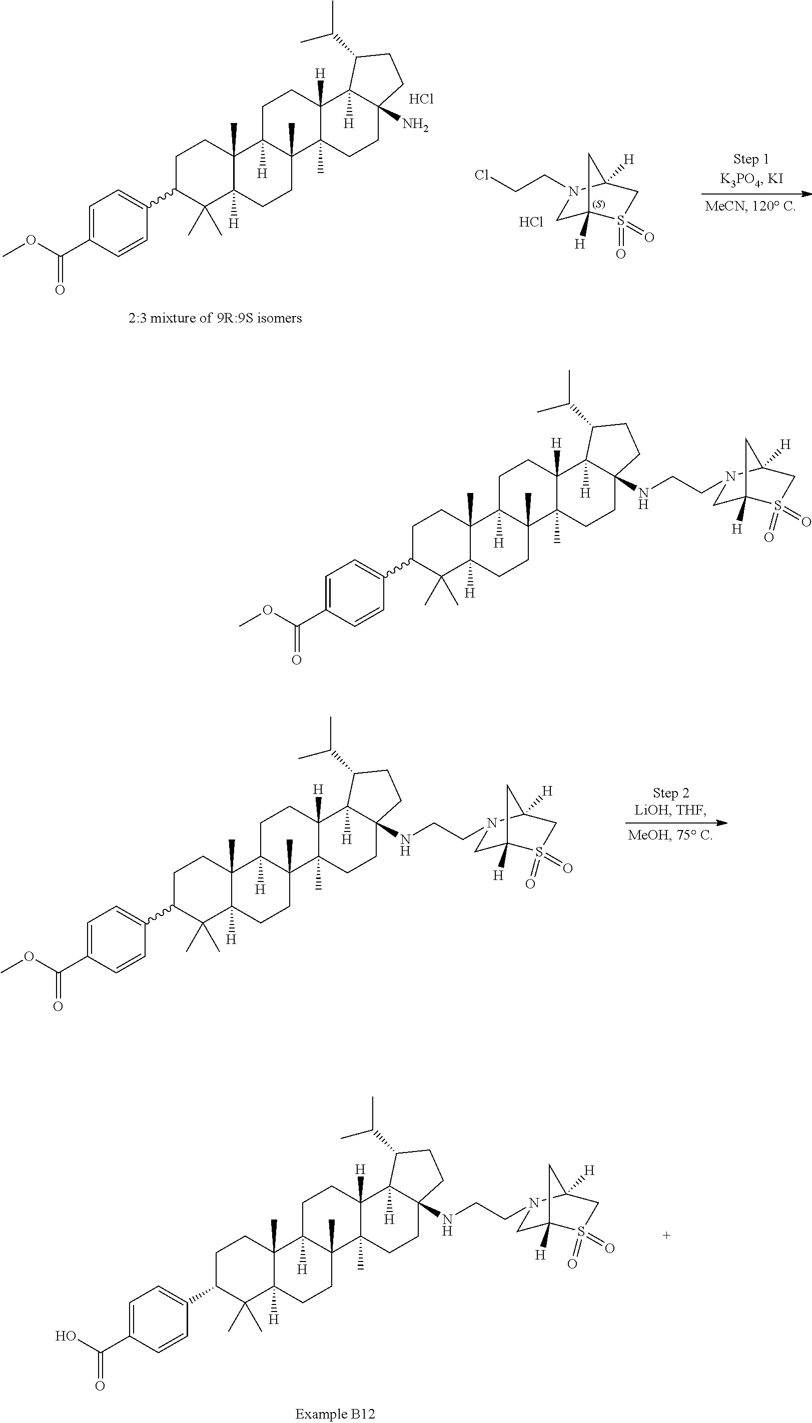

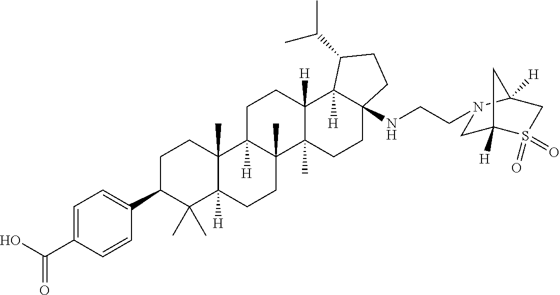

### Step 1: Preparation of methyl 4-((1S,3aS,5aR,5bR,7aS,11aS,11bR,13aR,13bR)-3a-((2-((1S,4S)-2,2-dioxido-2-thia-5-azabicyclo[2.2.1]heptan-5-yl)ethyl)amino)-1-isopropyl-5a,5b,8,8,11a-pentamethylicosahydro-1H-cyclopenta[a]chrysen-9-yl)benzoate

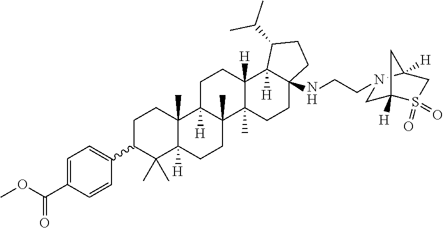

A 2:3 (9R:9S) isomeric mixture of ethyl 4-((1S,3aS,5aR,5bR,7aS,11aS,11bR,13aR,13bR)-3a-amino-1-isopropyl-5a,5b,8,8,8,11a-pentamethylicosahydro-1H-cyclopenta[a]chrysen-9-yl)benzoate HCl (0.350 g, 0.599 mmol), potassium iodide (0.212 g, 1.278 mmol), phosphoric acid potassium salt (0.542 g, 2.560 mmol), and (1S,4S)-5-(2-chloroethyl)-2-thia-5-azabicyclo[2.2.1]heptane 2,2-dioxide, HCl (0.315 g, 1.278 mmol) were combined in a 15 mL medium pressure tube and dried in a vacuum oven for 15 min and then flushed with N2(g). The solid mixture was charged with acetonitrile (6 mL) and the resulting suspension was heated to 120° C. After 5 h, the reaction was allowed to cool to rt and was subsequently treated with H₂O (25 mL) and extracted with 3×50 mL DCM. The combined organic layer was washed with brine, dried over MgSO₄, filtered and concentrated to brown solid. The crude material was purified by flash column chromatochromatography (40 g SiO₂, step elution 1:1 hex:EtOAc then 95:5 DCM:MeOH) and dried in vacuo to give methyl 4-((1S,3aS,5aR,5bR,7aS,11aS,11bR,13aR,13bR)-3a-((2-((1S,4S)-2,2-dioxido-2-thia-5-azabicyclo[2.2.1]heptan-5-yl)ethyl)amino)-1-isopropyl-5a,5b,8,8,11a-pentamethylicosahydro-1H-cyclopenta[a]chrysen-9-yl)benzoate (0.270 g, 0.374 mmol, 58.6% yield) as a brown solid isomeric mixture. LCMS: m/z=721.6 (M+H⁺), retention time 4.34 min (Method 5).

### Step 2. Preparation of 4-((1S,3aS,5aR,5bR,7aS,9R,11aS,11bR,13aR,13bR)-3a-((2-((1S,4S)-2,2-dioxido-2-thia-5-azabicyclo[2.2.1]heptan-5-yl)ethyl)amino)-1-isopropyl-5a,5b,8,8,11a-pentamethylicosahydro-1H-cyclopenta[a]chrysen-9-yl)benzoic acid

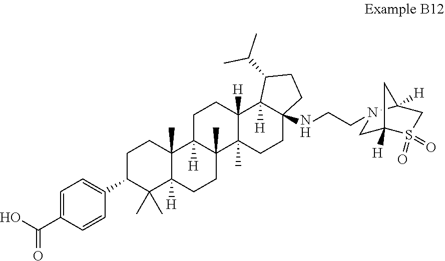

To a solution of the isomeric product from Step 1 methyl 4-((1S,3aS,5aR,5bR,7aS,11aS,11bR,13aR,13bR)-3a-((2-((1S,4S)-2,2-dioxido-2-thia-5-azabicyclo[2.2.1]heptan-5-yl)ethyl)amino)-1-isopropyl-5a,5b,8,8,11a-pentamethylicosahydro-1H-cyclopenta[a]chrysen-9-yl)benzoate (0.270 mg, 0.734 mmol) in THF (2 mL) and MeOH (1 mL) was added a solution of 3N lithium hydroxide (0.374 mL, 1.123 mmol) and the resulting mixture was stirred at 75° C. After 1.5 h, the reaction was allowed to cool to rt and was then purified twice by reverse phase preparative HPLC using Preparative Method 3 to give the desired title compound 4-((1S,3aS,5aR,5bR,7aS,9R,11aS,11bR,13aR,13bR)-3a-((2-((1S,4S)-2,2-dioxido-2-thia-5-azabicyclo[2.2.1]heptan-5-yl)ethyl)amino)-1-isopropyl-5a,5b,8,8,11a-pentamethylicosahydro-1H-cyclopenta[a]chrysen-9-yl)benzoic acid (0.0496 g, 14% yield) as a single isomer TFA salt. LCMS: m/z 707.6 (M+H⁺), 3.96 min (Method 4). ¹H NMR (400 MHz, 1:1 mixture of CDCl3 and MeOD, MeOD lock) δ ppm 7.90 (d, J=8.1 Hz, 2H), 7.27 (d, J=8.1 Hz, 2H), 3.97 (br. s., 1H), 3.65 (br. s., 1H), 3.15 (d, J=11.5 Hz, 2H), 3.05 (br. s., 3H), 2.99-2.89 (m, 1H), 2.83 (d, J=5.9 Hz, 1H), 2.59 (d, J=12.2 Hz, 1H), 2.40 (d, J=11.2 Hz, 1H), 2.05 (d, J=18.1 Hz, 2H), 2.00-1.82 (m, 4H), 1.81-1.63 (m, 7H), 1.62-1.44 (m, 9H), 1.41 (br. s., 1H), 1.39 (s, 3H), 1.24 (br. s., 2H), 1.12 (s, 3H), 1.09 (s, 6H), 0.96 (s, 3H), 0.90 (d, J=6.6 Hz, 3H), 0.81 (d, J=6.6 Hz, 3H), 0.54 (s, 3H).

### Example B13

### Preparation of 4-((1S,3aS,5aR,5bR,7aS,9R,11aS,11bR,13aR,13bR)-1-isopropyl-5a,5b,8,8,11a-pentamethyl-3a-((2-(4-(methylsulfonyl)piperidin-1-yl)ethyl)amino)icosahydro-1H-cyclopenta[a]chrysen-9-yl)benzoic acid

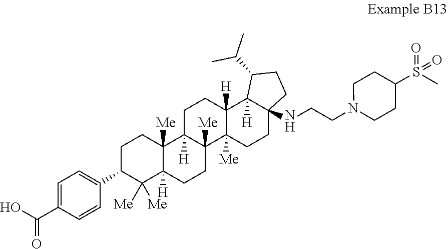

The title compound was prepared by a similar procedure as described in the preparation of 4-((1S,3aS,5aR,5bR,7aS,9R,11aS,11bR,13aR,13bR)-3a-((2-((1S,4S)-2,2-dioxido-2-thia-5-azabicyclo[2.2.1]heptan-5-yl)ethyl)amino)-1-isopropyl-5a,5b,8,8,11a-pentamethylicosahydro-1H-cyclopenta[a]chrysen-9-yl)benzoic acid, except 1-(2-chloroethyl)-4-(methylsulfonyl)piperidine, HCl was used in place of (1S,4S)-5-(2-chloroethyl)-2-thia-5-azabicyclo[2.2.1]heptane 2,2-dioxide hydrochloride in Step 1. Preparative HPLC purification of the crude Step 2 product using Preparative Method 4 gave 49.4 mg of the desired material as a TFA salt (6.4% yield over 2 steps). LCMS: m/z=723.5 (M+H)⁺, 2.29 min (Method 2). ¹H NMR (400 MHz, 1:1 mixture of CDCl3 and MeOD, MeOD lock) δ 7.96-7.88 (m, J=8.3 Hz, 2H), 7.34-7.27 (m, J=8.3 Hz, 2H), 3.24-3.02 (m, 5H), 2.94 (s, 4H), 2.83 (dd, J=9.0, 3.4 Hz, 1H), 2.71 (d, J=13.2 Hz, 1H), 2.51-2.37 (m, 1H), 2.28-2.12 (m, 3H), 2.11-1.97 (m, 4H), 1.95-1.80 (m, 4H), 1.79-1.54 (m, 10H), 1.54-1.47 (m, 5H), 1.47-1.28 (m, 5H), 1.28-1.22 (m, 1H), 1.20 (s, 3H), 1.10 (s, 6H), 0.98 (s, 3H), 0.92 (d, J=6.8 Hz, 3H), 0.83 (d, J=6.6 Hz, 3H), 0.55 (s, 3H).

Biology Data for the Examples
“μM” means micromolar;
“mL” means milliliter;
“μl” means microliter;
“mg” means milligram;
“μg” means microgram;

The materials and experimental procedures used to obtain the results reported in Table 1 are described below.

### HIV Cell Culture Assay—

MT-2 cells and 293T cells were obtained from the NIH AIDS Research and Reference Reagent Program. MT-2 cells were propagated in RPMI 1640 media supplemented with 10% heat inactivated fetal bovine serum, 100 μg/ml penicillin G and up to 100 units/ml streptomycin. The 293T cells were propagated in DMEM media supplemented with 10% heat inactivated fetal bovine serum (FBS), 100 units/ml penicillin G and 100 μg/ml streptomycin. The proviral DNA clone of NL₄₋₃ was obtained from the NIH AIDS Research and Reference Reagent Program. A recombinant NL₄₋₃ virus, in which a section of the nef gene from NL₄₋₃ was replaced with the Renilla luciferase gene, was used as a reference virus. In addition, residue Gag P373 was converted to P373S. Briefly, the recombinant virus was prepared by transfection of the altered proviral clone of NL₄₋₃. Transfections were performed in 293T cells using LipofectAMINE PLUS from Invitrogen (Carlsbad, Calif.), according to manufacturer's instruction. The virus was titered in MT-2 cells using luciferase enzyme activity as a marker. Luciferase was quantitated using the Dual Luciferase kit from Promega (Madison, Wis.), with modifications to the manufacturer's protocol. The diluted Passive Lysis solution was pre-mixed with the re-suspended Luciferase Assay Reagent and the re-suspended Stop & Glo Substrate (2:1:1 ratio). Fifty (50) μL of the mixture was added to each aspirated well on assay plates and luciferase activity was measured immediately on a Wallac TriLux (Perkin-Elmer). Antiviral activities of inhibitors toward the recombinant virus were quantified by measuring luciferase activity in cells infected for 4-5 days with NLRluc recombinants in the presence serial dilutions of the inhibitor. The EC₅₀ data for the compounds is shown in Table 1. Note that some of the data is provided in abbreviated exponential form such that, for example, 2.53E−3, is equivalent to 2.53×10⁻³.

TABLE 1

| Example | | EC₅₀ |
|---|---|---|
| # | Structure | (μM) |
| B1 | 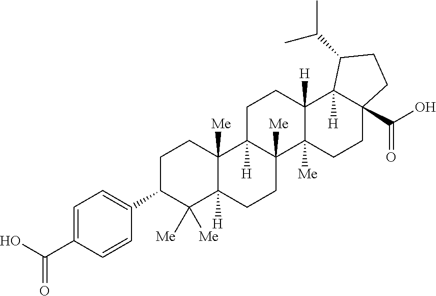 | 0.01 |
| B2 | 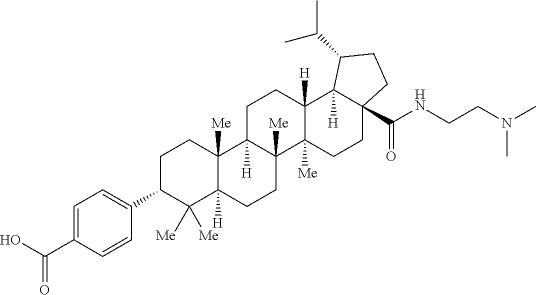 | 2.53E−03 |
| B3 | 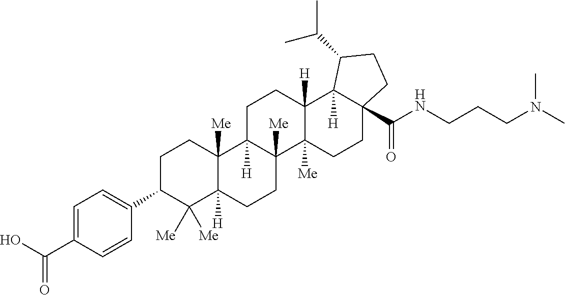 | 2.33E−03 |
| B4 | 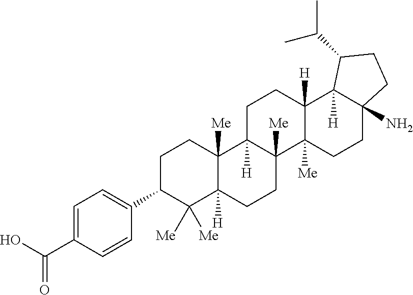 | 4.53E−03 |
| B5 | 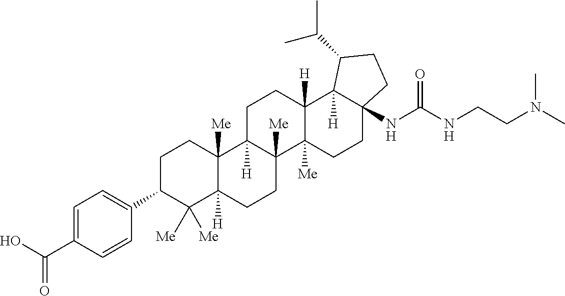 | 5.16E−03 |
| B6 | 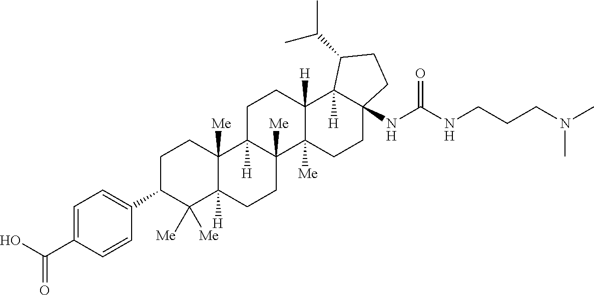 | 5.13E−03 |
| B7 | 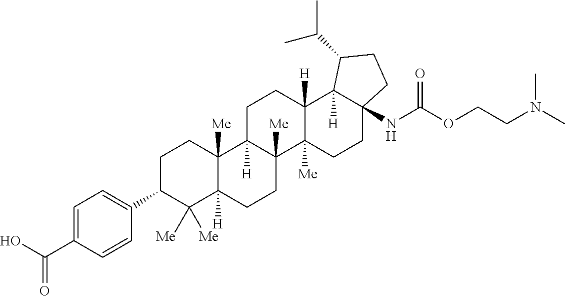 | 6.45E−03 |
| B8 | 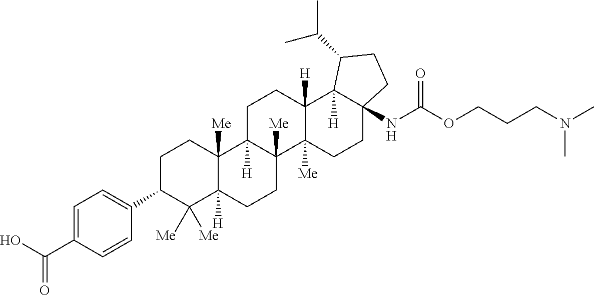 | 4.49E−03 |
| B9 | 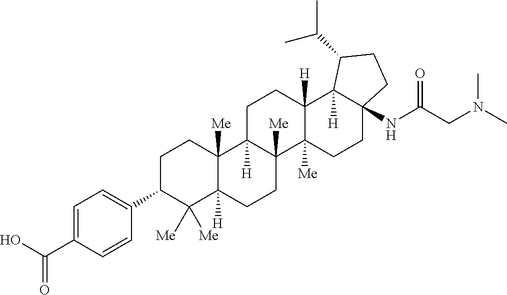 | 0.01 |
| B10 | 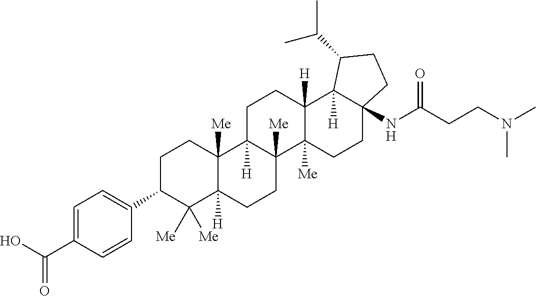 | 3.81E−03 |
| B11 | 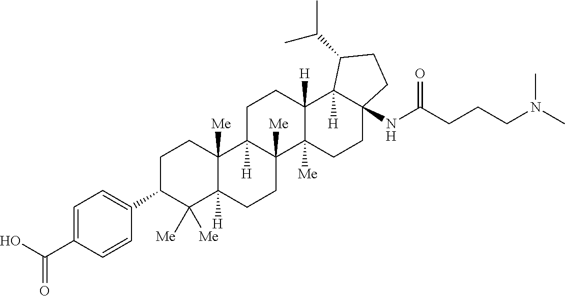 | 5.94E−03 |
| B12 | 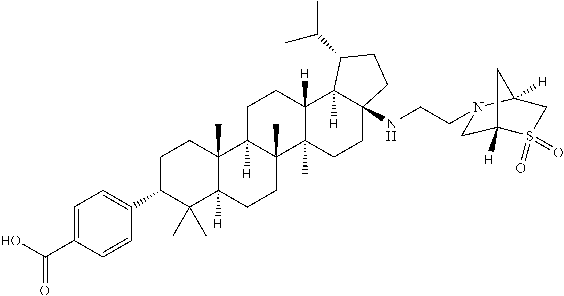 | 6.05E−04 |
| B13 | 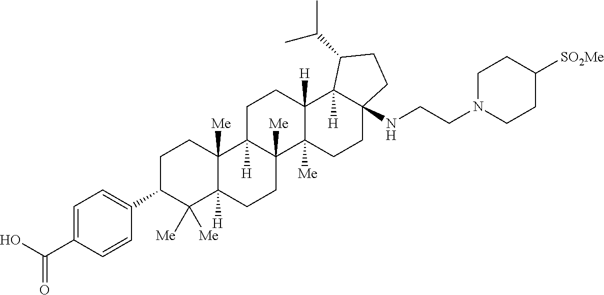 | 1.58E−03 |

The disclosure is not limited to the foregoing illustrative examples and the examples should be considered in all respects as illustrative and not restrictive, reference being made to the appended claims, rather than to the foregoing examples, and all changes which come within the meaning and range of equivalency of the claims are therefore intended to be embraced.

1. A compound of Formula I: 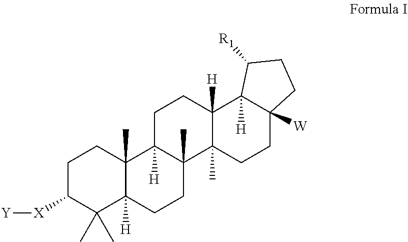
or a pharmaceutically acceptable salt thereof: wherein;
R₁ is isopropenyl or isopropyl;
X is a phenyl or heteroaryl ring substituted with A, wherein A is at least one member selected from —H, -halo, -hydroxyl, —C₁₋₆ alkyl, —C₁₋₆ alkoxy, and —COOR₂;
R₂ is —H, —C₁₋₆ alkyl, -alkylsubstituted C₁₋₆ alkyl or -arylsubstituted C₁₋₆ alkyl;
Y is selected from —COOR₂, —C(O)NR₂SO₂R₃, —C(O)NHSO₂NR₂R₂, —NR₂SO₂R₂, —SO₂NR₂R₂, —C₃₋₆ cycloalkyl-COOR₂, —C₂₋₆ alkenyl-COOR₂, —C₂₋₆ alkynyl-COOR₂, —C₁₋₆ alkyl-COOR₂, —NHC(O)(CH₂)ₙ—COOR₂, —SO₂NR₂C(O)R₂, -tetrazole, and —CONHOH,
wherein n=1-6;
R₃ is —C₁₋₆ alkyl or -alkylsubstituted C₁₋₆ alkyl;
W is selected from —CH₂OR₂, —COOR₂, —NR₄R₅, —CONR₂₆R₂₇, —CH₂NR₂₆R₂₇, —NR₄COR₆, —NR₄C(O)NR₄R₅, and —NR₄COOR₆;
R₄ is selected from —H, —C₁₋₆ alkyl, —C₁₋₆ alkyl-C(OR₃)₂—C₃₋₆ cycloalkyl, —C₁₋₆ substituted alkyl, —C₁₋₆ alkyl-C₃₋₆ cycloalkyl, —C₁₋₆ alkyl-Q₁, —C₁₋₆ alkyl-C₃₋₆ cycloalkyl-Q₁, aryl, heteroaryl, substituted heteroaryl, —COR₆, —COCOR₆, —SO₂R₇, —SO₂NR₂R₂,
wherein Q₁ is selected from C₃₋₁₀ carbocycle, substituted C₃₋₁₀ carbocycle, C₃₋₁₀ heterocycle, substituted C₃₋₁₀ heterocycle, aryl, heteroaryl, substituted heteroaryl, halogen, —CF₃, —OR₂, —COOR₂, —NR₈R₉, —CONR₁₀R₁₁ and —SO₂R₇;
R₅ is selected from —H, —C₁₋₆ alkyl, —C₃₋₆ cycloalkyl, —C₁₋₆ alkylsubstituted alkyl, —C₁₋₆ alkyl-NR₈R₉, —COR₆, —COCOR₆, —SO₂R₇ and —SO₂NR₂R₂;
with the proviso that only one of R₄ or R₅ can be selected from —COR₆, —COCOR₆, —SO₂R₇ and —SO₂NR₂R₂;
R₆ is selected from —H, —C₁₋₆ alkyl, —C₁₋₆ alkyl-substitutedalkyl, —C₃₋₆ cycloalkyl, —C₃₋₆ substitutedcycloalkyl-Q₂, —C₁₋₆ alkyl-Q₂, —C₁₋₆ alkyl-substitutedalkyl-Q₂, —C₃₋₆ cycloalkyl-Q₂, aryl-Q₂, —NR₁₃R₁₄, and —OR₁₅;
wherein Q₂ is selected from C₃₋₁₀ carbocycle, substituted C₃₋₁₀ carbocycle, C₃₋₁₀ heterocycle, substituted C₃₋₁₀ heterocycle, aryl, heteroaryl, substituted heteroaryl, —OR₂, —COOR₂, —NR₈R₉, SO₂R₇, —CONHSO₂R₃, and —CONHSO₂NR₂R₂;
R₇ is selected from —C₁₋₆ alkyl, —C₁₋₆ substituted alkyl, —C₃₋₆ cycloalkyl, aryl, and heteroaryl;
R₈ and R₉ are independently selected from —H, —C₁₋₆ alkyl, —C₁₋₆ substituted alkyl, aryl, heteroaryl, substituted aryl, substituted heteroaryl, —C₁₋₆ alkyl-Q₂, and —COOR₃,
or R₈ and R₉ are taken together with the adjacent N to form a cycle selected from: 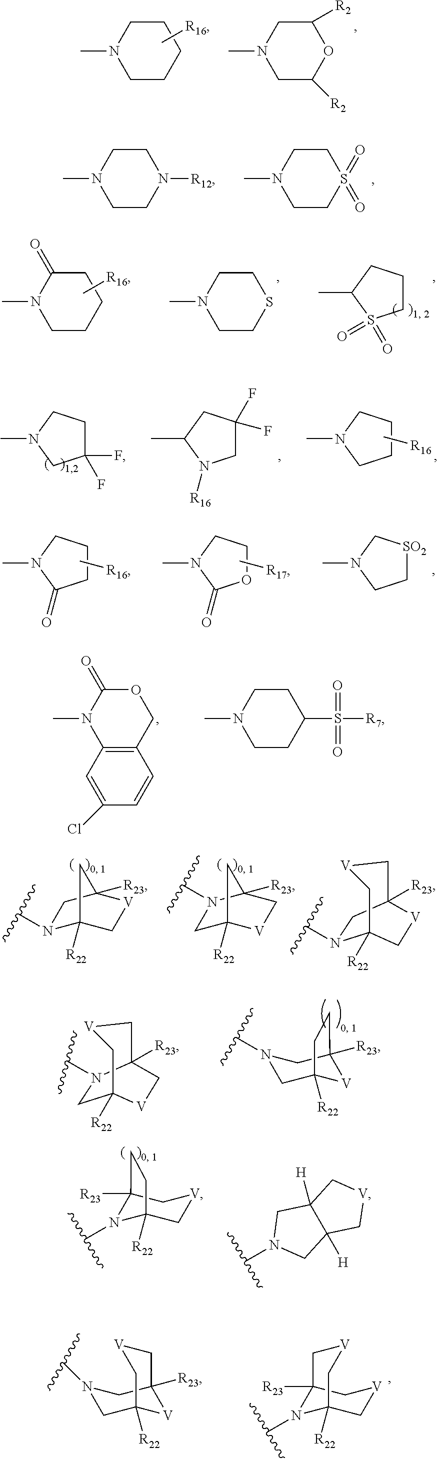 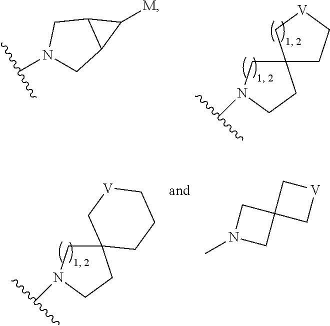
V is selected from —CR₂₄R₂₅, —SO₂, —O and —NR₁₂;
M is selected from —CHR₂₄R₂₅, —NR₂₆R₂₇, —SO₂R₇, —SO₂NR₃R₃ and —OH;
with the proviso that only one of R₈ or R₉ can be —COOR₃;
R₁₀ and R₁₁ are independently selected from —H, —C₁₋₆ alkyl, —C₁₋₆ substituted alkyl and —C₃₋₆ cycloalkyl,
or R₁₀ and R₁₁ are taken together with the adjacent N to form a cycle such as 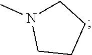
R₁₂ is selected from —C₁₋₆ alkyl, —C₁₋₆ alkyl-OH; —C₁₋₆ alkyl, —C₁₋₆ substituted alkyl, —C₃₋₆ cycloalkyl, —COR₇, —COONR₂₂R₂₃, —SOR₇, and —SONR₂₄R₂₅;
R₁₃ and R₁₄ are independently selected from —H, —C₁₋₆ alkyl, —C₃₋₆ cycloalkyl, —C₁₋₆ substituted alkyl, —C₁₋₆ alkyl-Q₃, —C₁₋₆ alkyl-C₃₋₆ cycloalkyl-Q₃ and C₁₋₆ substituted alkyl-Q₃,
or R₁₃ and R₁₄ are taken together with the adjacent N to form a cycle selected from: 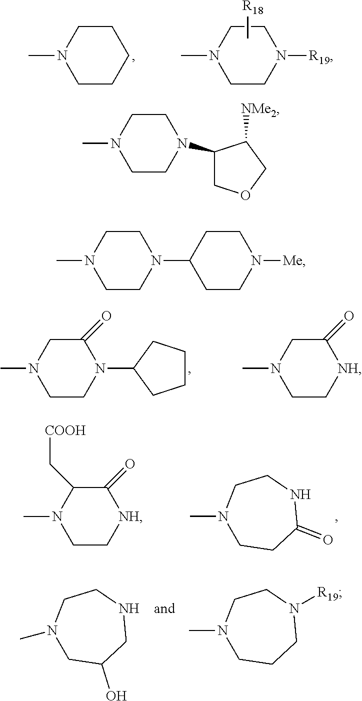
Q₃ is selected from heteroaryl, substituted heteroaryl, —NR₂₀R₂₁, ⁻CONR₂R₂, —COOR₂, —OR₂, and —SO₂R₃;
R₁₅ is selected from —C₁₋₆ alkyl, —C₃₋₆ cycloalkyl, —C₁₋₆ substituted alkyl, —C₁₋₆ alkyl-Q₃, —C₁₋₆ alkyl-C₃₋₆ cycloalkyl-Q₃ and —C₁₋₆ substituted alkyl-Q₃;
R₁₆ is selected from —H, —C₁₋₆ alkyl, —NR₂R₂, and —COOR₃;
R₁₇ is selected from —H, —C₁₋₆ alkyl, —COOR₃, and aryl;
R₁₈ is selected from —COOR₂ and —C₁₋₆ alkyl-COOR₂;
R₁₉ is selected from —H, —C₁₋₆ alkyl, —C₁₋₆ alkyl-Q₄, —COR₃, —COOR₃,
wherein Q₄ is selected from —NR₂R₂ and —OR₂;
R₂₀ and R₂₁ are independently selected from —H, —C₁₋₆ alkyl, —C₁₋₆ substituted alkyl, —C₁₋₆ substituted alkyl-OR₂, and —COR₃,
or R₂₀ and R₂₁ are taken together with the adjacent N to form a cycle selected from 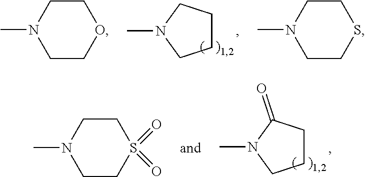
with the proviso that only one of R₂₀ or R₂₁ can be —COR₃;
R₂₂ and R₂₃ are independently selected from H, —C₁₋₆ alkyl, —C₁₋₆ substituted alkyl, and —C₁₋₆ cycloalkyl,
or R₂₂ and R₂₃ are taken together with the adjacent N to form a cycle selected from 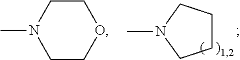
R₂₄ and R₂₅ are independently from the group of H, —C₁₋₆ alkyl, —C₁₋₆ substituted alkyl, —C₁₋₆ alkyl-Q₅, —C₁₋₆ cycloalkyl, aryl, substituted aryl, heteroaryl, and substituted heteroaryl, and Q₅ is selected from halogen and SO₂R₃,
R₂₆ and R₂₇ are independently selected from —H, —C₁₋₆ alkyl, —C₁₋₆ substituted alkyl, aryl, heteroaryl, substituted aryl, substituted heteroaryl, —C₁₋₆ alkyl-Q₂,
or R₂₆ and R₂₇ are taken together with the adjacent N to form a cycle selected from:

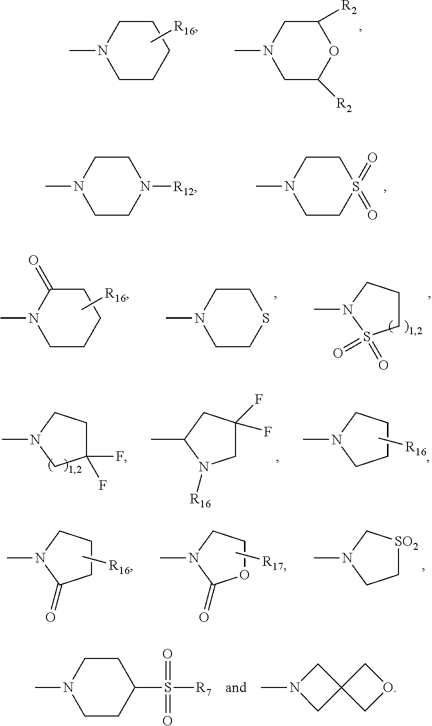

2. (canceled)

3. A compound or salt as claimed in claim 1, wherein X is phenyl.

4. (canceled)

5. A compound or salt as claimed in claim 3, wherein Y is —COOH.

6-11. (canceled)

12. A compound or salt as claimed in claim 5 wherein W is —CH₂OR₂.

13. A compound or salt as claimed in claim 5 wherein W is —COOR₂.

14. A compound or salt as claimed in claim 5 wherein W is —COOH.

15. A compound or salt as claimed in claim 5 wherein W is —NR₄R₅.

16. A compound or salt as claimed in claim 5 wherein W is —CONR₂₆R₂₇.

17. A compound or salt as claimed in claim 5 wherein W is —CH₂NR₂₆R₂₇.

18. A compound or salt as claimed in claim 5 wherein W is —NR₄COR₆.

19. A compound or salt as claimed in claim 5 wherein W is —NR₄C(O)NR₄R₅.

20. A compound or salt as claimed in claim 5 wherein W is —NR₄COOR₆.

21. A pharmaceutical composition comprising a compound or salt of claim 1 and a pharmaceutically acceptable carrier.

22-23. (canceled)

24. A method for treating HIV infection comprising administering a compound or salt of claim 1, or a pharmaceutically acceptable salt thereof, to a patient in need thereof.

25-27. (canceled)
